(19) United States
(12) Patent Application Publication
(10) Pub. No.: US 2024/0426847 A1
(43) Pub. Date: Dec. 26, 2024

(54) COMPOSITIONS AND METHODS FOR THE DETERMINATION OF SODIUM CONCENTRATION

(71) Applicant: The University of Chicago, Chicago, IL (US)

(72) Inventors: Yamuna Krishnan, Chicago, IL (US); Junyi Zou, Changsha (CN)

(21) Appl. No.: 18/743,714

(22) Filed: Jun. 14, 2024

Related U.S. Application Data
(60) Provisional application No. 63/521,492, filed on Jun. 16, 2023.

(51) Int. Cl. G01N 33/84; G01N 33/53; G01N 33/58

(52) U.S. Cl. CPC G01N 33/84; G01N 33/5308; G01N 33/582

(57) ABSTRACT

This disclosure relates to methods for determining sodium concentration in biological samples. More particularly, this disclosure relates to methods capable of determining Na⁺ concentration using nucleic acid complexes.

### CROSS-REFERENCE TO RELATED APPLICATIONS

This application claims the benefit of priority of U.S. Provisional Application No. 63/521,492, filed Jun. 16, 2023, which is incorporated herein by reference in its entirety.

### REFERENCE TO AN ELECTRONIC SEQUENCE LISTING

This application contains a Sequence Listing which has been submitted electronically and is hereby incorporated by reference in its entirety. The Sequence Listing was created on May 31, 2024, is named “23-0476-US_SequenceListing_ST26” and is 14,781 bytes in size.

### BACKGROUND

### Field of Disclosure

This disclosure relates to methods for determining sodium concentration in biological samples. More particularly, this disclosure relates to methods capable of determining Na⁺ concentration using nucleic acid complexes.

### Technical Background

Cellular sodium ion (Na⁺) homeostasis regulates organism physiology1 2,3. However, the current understanding is limited to Na⁺ mobilization at the plasma membrane. Growing evidence suggests that organelles, which share a cytosol with the plasma membrane, may also participate in Na⁺ homeostasis4,5. The presence of membrane potential and the abundance of Na⁺ channels and transporters6-13 in organelles suggest vigorous Na⁺ transport across organelle membranes. However, precisely how organelles contribute to Na⁺ homeostasis is unknown because Na⁺ cannot be imaged at sub-cellular resolution.

Organelle membranes comprise ˜95% of total membrane in the cell, yet most of the understanding of cellular Na⁺ homeostasis relates to the 2-5% equivalent to the plasma membrane14. Few lines of evidence now suggest that organelles could contribute to the metabolism and mobilization of Na⁺ in single cells. In yeast, for example, many endosomal Na⁺/H⁺ exchangers were identified due to the lethality they caused upon salt stress when they were knocked out11,15. Even in higher organisms, the loss of a vacuolar Na⁺/H⁺ exchanger (Nhx1) in Japanese morning glory or a lysosomal Na⁺/Ca²⁺/K⁺ transporter (slc24a5) in zebrafish cause striking pigmentation phenotypes4,5. Apart from transporters, organelles have several voltage-gated Na⁺ channels that are likely functional since many endocytic organelles were recently found to harbor membrane potential6,7,12,16. Although extracellular and cytosolic Na⁺ at the tissue and single cell levels are known1-3.17, those within organelles are not. Consequently, the direction of ion flow when organelle-resident Na⁺ channels or transporters are activated and therefore how organelles might contribute to cellular Na⁺ homeostasis in health and disease cannot be predicted.

Lumenal Na⁺ levels in organelles have not been mapped because there are no probes that work in acidic organelles. All fluorescent Na⁺ probes are acid sensitive because they detect Na⁺ by coordination via protonatable groups18. Further, genetically encodable reporters of Na⁺ do not exist. Hence, previous estimates of lumenal Na⁺ relied on elemental analysis of isolated organelles or null point titration that averages the information from different organelles19,20.7. Since lumenal ionic composition and membrane potential of endocytic organelles vary greatly21,22, population-averaged measurements mask the precise contribution of organelles to Na⁺ homeostasis6,23.

Accordingly, there is a need to develop compositions and methods that can be used to measure Na⁺ concentrations in a pH-independent manner.

### SUMMARY

In one aspect, the present disclosure provides for a method for determining a Na⁺ concentration in a sample comprising:
providing a nucleic acid complex comprising:
a first single-stranded nucleic acid molecule comprising a Na+ fluorophore linked thereto, wherein the intensity of the fluorescence of the Na+ fluorophore is related to the Na+ concentration; and
a second single-stranded nucleic acid molecule that is partially or fully complementary to the first single-stranded molecule,
measuring an intensity of the Na+ fluorophore fluorescence; and
determining the Na+ concentration from the intensity.

In another aspect, the present disclosure provides for a nucleic acid complex comprising:
a first single-stranded nucleic acid molecule comprising a Na⁺ fluorophore linked thereto, wherein the intensity of the fluorescence of the Na⁺ fluorophore is related to the Na⁺ concentration; and
a second single-stranded nucleic acid molecule that is partially or fully complementary to the first single-stranded molecule.

In another aspect, the present disclosure provides for a compound having the structure:

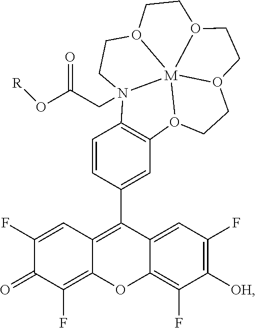

wherein:
M is an alkali metal ion, or is absent; and
R is H, C₁-C₁₂ alkyl, (C₁-C₁₂ alkyl)-SH, (C₁-C₁₂ alkyl)-N₃, C₂-C₁₂ alkenyl group, or C₂-C₁₂ alkynyl.

Other aspects of the disclosure will be apparent to those skilled in the art in view of the description that follows.

### BRIEF DESCRIPTION OF THE DRAWINGS

The accompanying drawings are included to provide a further understanding of the systems and methods of the disclosure, and are incorporated in and constitute a part of this specification. The drawings are not necessarily to scale, and sizes of various elements may be distorted for clarity. The drawings illustrate one or more embodiment(s) of the disclosure and together with the description serve to explain the principles and operation of the disclosure.

FIGS. 1A-1E illustrate that RatiNa is a ratiometric, pH-independent and specific reporter of Na⁺.

FIGS. 2A-2E illustrate in cell and in vivo calibration of RatiNa to measure lysosomal Na⁺.

FIGS. 3A-3C illustrate that RatiNa captures physiological changes in organellar Na⁺.

FIGS. 4A-4E illustrate that lysosomal Na⁺ transport is vital for salt adaptation in C. elegans.

FIGS. 5A-5G illustrate that Chicago Green (CG) is pH insensitive and selective to Na⁺ prior to incorporation into RatiNa.

FIGS. 6A-6G illustrate that calibration of RatiNa and its stability in lysosomes of RAW264.7 macrophages.

FIGS. 7A-7B illustrate specificity of RatiNa targeting to endocytic organelles.

FIGS. 8A-8C illustrate that Na⁺ transporter mutant worms adapt differently to salt stress.

FIGS. 9A-9D illustrate that lysosomal Na⁺ levels in various Na⁺ transporter mutants increase upon salt adaptation.

FIGS. 10A-10B illustrate the synthesis of Chicago Green.

FIGS. 11A-11D illustrate the conjugation of CG to DNA.

FIGS. 12A-12B illustrate the validation of CG-DNA conjugate efficiency and Na⁺ sensitivity.

FIGS. 13A-13B illustrate the gel electrophoresis of RatiNa sensors.

FIGS. 14A-14B illustrate the bead calibration of RatiNa sensors.

FIG. 15 illustrates a workflow of brood size assay for salt adaptation.

FIG. 16 illustrates a model of NHX-5 switching direction of Na⁺ transport during salt adaptation.

FIG. 17 illustrates a design of RatiNa sensors.

### DETAILED DESCRIPTION

Herein, a pH-independent, organelle-targetable, ratiometric probe is described that reports lumenal Na⁺. It is a DNA nanodevice containing a Na⁺-sensitive fluorophore, a reference dye and an organelle targeting domain. By measuring Na⁺ at single endosome resolution in mammalian cells and in C. elegans, it was discovered that Na⁺ levels in endocytic organelles exceed cytosolic levels and, unlike any other ion mapped so far, lumenal Na⁺ decreases as endosomes mature. Further, it was found that nematodes adapt to salt stress by changing their lysosomal Na⁺ levels and that NHX-5, a lysosomal Na⁺ transporter, is vital for adaptation. This reveals a role for lysosomes in metazoan Na⁺ metabolism. The newfound ability to image sub-cellular Na⁺ will unveil mechanisms of Na⁺ transport and metabolism at an entirely new level of cellular detail.

In one aspect, the present disclosure relates to a method for determining a Na⁺ concentration in a sample. The method comprises providing a nucleic acid complex, measuring an intensity of the Na⁺ fluorophore fluorescence, and determining the Na⁺ concentration from the intensity. In various embodiments as otherwise described herein, the nucleic acid complex comprises a first single-stranded nucleic acid molecule comprising a Na⁺ fluorophore linked thereto, wherein the intensity of the fluorescence of the Na⁺ fluorophore is related to the Na⁺ concentration; and a second single-stranded nucleic acid molecule that is partially or fully complementary to the first single-stranded molecule.

In various embodiments, the sample may be a biological sample selected from a cell, cell extract, cell lysate, tissue, tissue extract, bodily fluid, serum, blood, and blood product. In various other embodiments, the sample may be a live cell.

In various embodiments, determining the Nat concentration may be in early endosome, late endosome, plasma membrane, lysosome, autophagolysosome, recycling endosome, cis Golgi network, trans Golgi network, endoplasmic reticulum, peroxisomes, or secretory vesicles.

In various embodiments, the nucleic acid complex may further comprise a reference fluorophore linked to the first single-stranded nucleic acid molecule or the second single-stranded nucleic acid molecule. In various other embodiments, the nucleic acid complex may further comprise a reference fluorophore linked to the second single-stranded nucleic acid.

In various embodiments, the reference fluorophore may comprise an Atto dye, an Alexa Fluor® dye, a Cy® dye, or a BODIPY dye. In various other embodiments, the reference fluorophore may comprise an ATTO647 fluorophore.

In various embodiments, the reference fluorophore may be pH insensitive and Na⁺ concentration insensitive, within physiological ranges.

In various embodiments, the method may further comprise measuring the intensity of the reference fluorescence of the reference fluorophore, and normalizing the Na⁺ fluorescence to the reference fluorescence.

In various embodiments, the Na⁺ fluorophore may be pH insensitive.

In various embodiments, the Na⁺ fluorophore may comprise a 1-aza-15-crown-5 ether moiety.

In various embodiments, the Na⁺ fluorophore may comprise

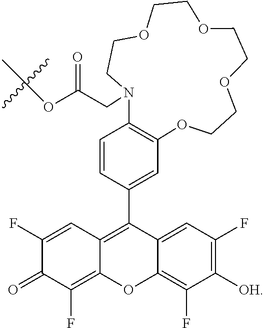 [SMILES: *OC(=O)CN1CCOCCOCCOCCOc2cc(-c3c4cc(F)c(=O)c(F)c-4oc4c(F)c(O)c(F)cc34)ccc21]

In various embodiments, the Na⁺ fluorophore may be linked to the first single-stranded nucleic acid through a linker moiety stable under physiological conditions. In various other embodiments, the Na⁺ fluorophore may be linked to the first single-stranded nucleic acid molecule through a triazole, thioether, or alkenyl sulfide group.

In various embodiments, the Na⁺ fluorophore may further comprise a linker moiety configured to form the triazole, thioether, or alkenyl sulfide group through a reaction of an azide, alkyne, or thiol moiety on the Na⁺ fluorophore and an azide, alkyne or alkene moiety on the first single-stranded nucleic acid molecule, as chemically appropriate.

In various embodiments, the Na⁺ fluorophore may comprise

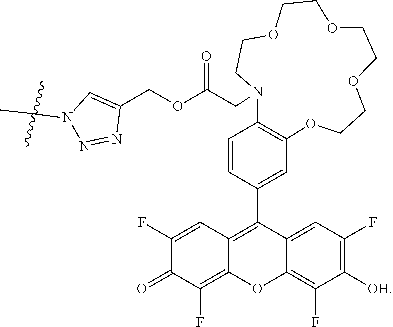 [SMILES: *n1cc(COC(=O)CN2CCOCCOCCOCCOc3cc(-c4c5cc(F)c(=O)c(F)c-5oc5c(F)c(O)c(F)cc45)ccc32)nn1]

In various embodiments, the nucleic acid complex may further comprise a third single-stranded nucleic acid molecule that is partially complementary to the second single-stranded molecule.

In various embodiments, the nucleic acid complex may further comprise a targeting moiety. In various embodiments as otherwise described herein, the targeting moiety may be a nucleic acid sequence. In various other embodiments, the targeting moiety may have a cognate artificial protein receptor.

In various embodiments, the targeting moiety may be selected from an aptamer, a duplex domain targeted to an artificial protein receptor, a nucleic acid sequence that binds an anionic-ligand binding receptor, and an endocytic ligand.

In various embodiments, the targeting moiety may comprise a peptide directly or indirectly conjugated to the nucleic acid molecule.

In various embodiments, the targeting moiety may comprise one or more of a fusogenic peptide, a membrane-permeabilizing peptide, a sub-cellular localization sequence, or a cell-receptor ligand. In various embodiments as otherwise described herein, the targeting moiety may comprise a sub-cellular localization sequence, and the sub-cellular localization sequence may target the nucleic acid complex to a region of a cell where spatial localization of a targeted protein is present. In various embodiments, the sub-cellular localization sequence may target the nucleic acid complex to a region of the cell selected from the group consisting of the cytosol, the endoplasmic reticulum, the mitochondrial matrix, the chloroplast lumen, the medial trans-Golgi cistemae, the lumen of lysosome, the lumen of an endosome, the peroxisome, the nucleus, and a specific spatial location on the plasma membrane.

In various embodiments, the targeting moiety may be encoded on the same nucleic acid strand as the first single-stranded nucleic acid molecule, the second single-stranded nucleic acid molecule, the third single-stranded nucleic acid molecule, or any combination thereof. In various embodiments as otherwise described herein, the targeting moiety may be located on the third single-stranded nucleic acid molecule.

In various embodiments, the first and/or second single-stranded nucleic acid molecule may be less than 200 nucleotides; or less than 100 nucleotides; or less than 50 nucleotides. In various other embodiments, the first and third single-stranded nucleic molecules together may be the same length as the second single-stranded nucleic acid molecule.

In various embodiments, the determined Na⁺ concentration may be in a range of 10 μM to 500 mM. In various other embodiments, the determined Na⁺ concentration may be in a range of 100 μM to 150 mM. In various embodiments, the determined Na⁺ concentration may be in a range of 1 mM to 150 mM.

In various embodiments, the first single-stranded nucleic acid molecule may have the sequence 5′-CG-ATC AAC ACT GCA TAT ATA TAC GAC C-3′ [SEQ ID NO: 1] or 5′-ATC AAC ACT GCA TAT ATA TAC GAC C-3′ [SEQ ID NO:2]; and the second single-stranded nucleic acid molecule may have the sequence 5′-ATTO647N—C ACT GCA CAC CAG ACA GCA A G GTC GTA TAT ATA TGC AGT GTT GAT-3′ [SEQ ID NO:3].

In various embodiments, the nucleic acid complex may further comprise a third single-stranded nucleic acid molecule that is partially complementary to the second single-stranded molecule, and wherein the third single-stranded nucleic acid molecule may have the sequence 5′-T TGC TGT CTG GTG TGC AGT G-BioTEG-3′ [SEQ ID NO:4] or 5′-T TGC TGT CTG GTG TGC AGT G-3′ [SEQ ID NO:5], wherein BioTEG is a biotin-triethylene glycol moiety.

In another aspect, the present disclosure relates to a nucleic acid complex as described herein.

In various embodiments, the nucleic acid complex may comprise a first single-stranded nucleic acid molecule comprising a Na⁺ fluorophore linked thereto, wherein the intensity of the fluorescence of the Na⁺ fluorophore is related to the Na⁺ concentration; and a second single-stranded nucleic acid molecule that is partially or fully complementary to the first single-stranded molecule, as described herein.

In another aspect, the present disclosure relates to a compound having the structure

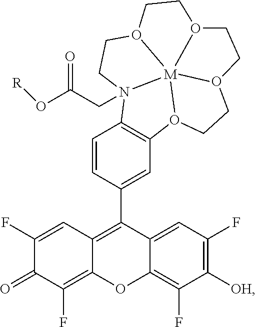

wherein:
M is an alkali metal ion, or is absent; and
R is H, C₁-C₁₂ alkyl, (C₁-C₁₂ alkyl)-SH, (C₁-C₁₂ alkyl)-N₃, C₂-C₁₂ alkenyl group, or C₂-C₁₂ alkynyl.

In various embodiments, M is Na⁺ or absent.

In various embodiments, R is C₂-C₆ alkynyl.

In various embodiments, R is —CH₂—C≡CH.

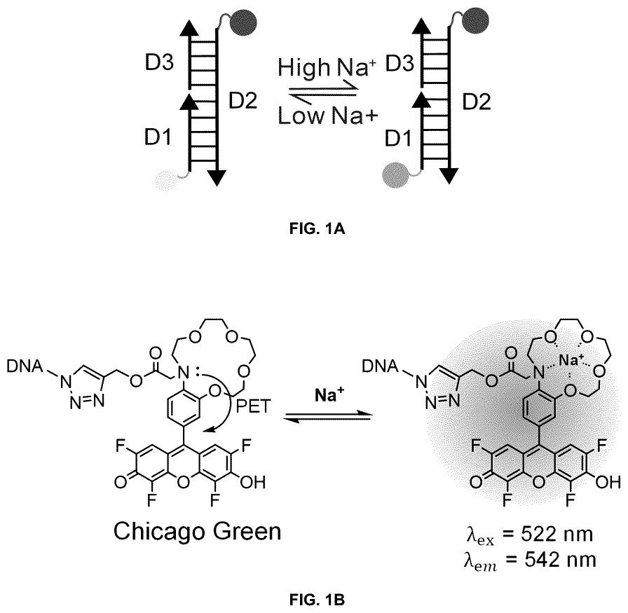

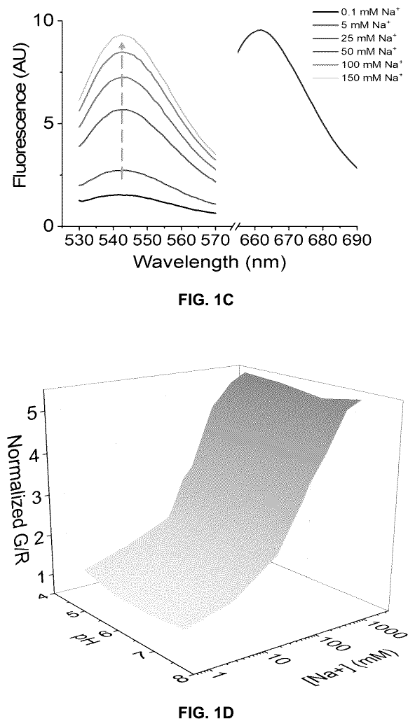

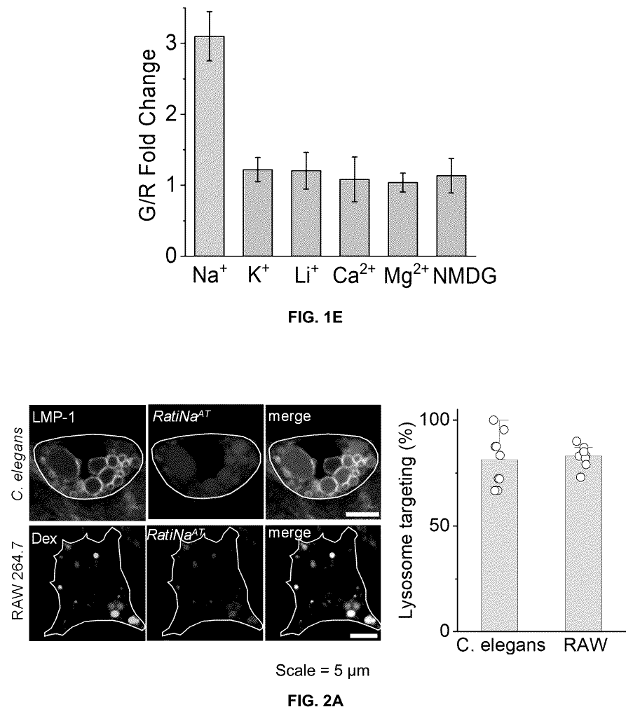

FIGS. 1A-1E illustrate that RatiNa is a ratiometric, pH-independent and specific reporter of Na⁺. FIG. 1A is a schematic of the three ssDNA strands in RatiNa: D1 displays a Na⁺ sensing dye, Chicago Green (CG, green circle), D2 bears a reference ATTO647 fluorophore (red circle) and D3 harbors an organelle targeting motif. Na⁺ binding makes CG fluoresce (dark green circle). D1 and D3 are complementary to D2. FIG. 1B shows working principle of CG, the pH insensitive yet Na⁺ sensitive dye. CG is quenched by the N lone pair on the aza-crown ether via photoinduced electron transfer (left). Na⁺ binding relieves quenching and turns on the fluorescence (right). FIG. 1C shows that RatiNa ratiometrically reports Na⁺. Increasing Na⁺ increases fluorescence of CG (green traces) but not that of ATTO647 (red trace). FIG. 1D shows that in vitro calibration profile of RatiNa on beads as a function of Na⁺ and PH levels. Na⁺ response of RatiNa is unaffected from pH 4.5 to 7.4. FIG. 1E shows that RatiNa responds specifically to Na⁺ and not to other physiologically relevant cations. Na⁺, K⁺=145 mM; Li⁺, Ca²⁺, Mg²⁺=10 mM; NMDG=0.3 M.

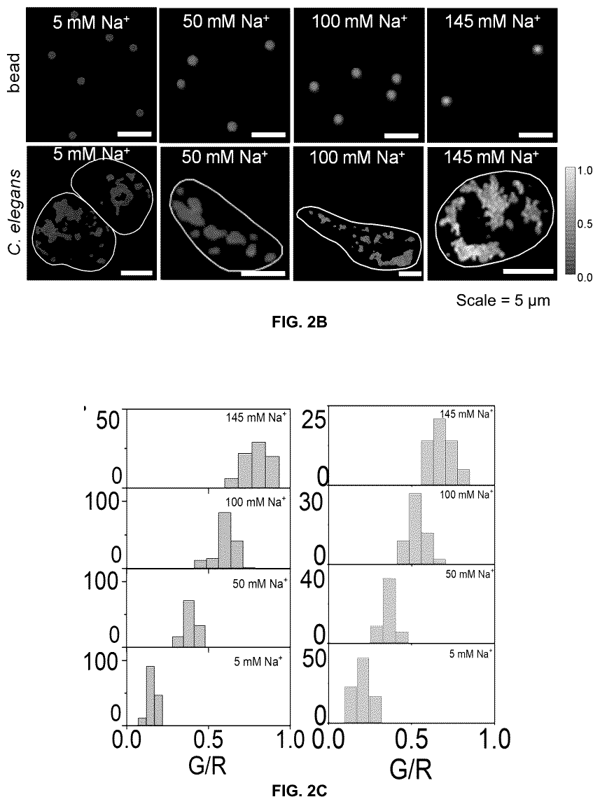

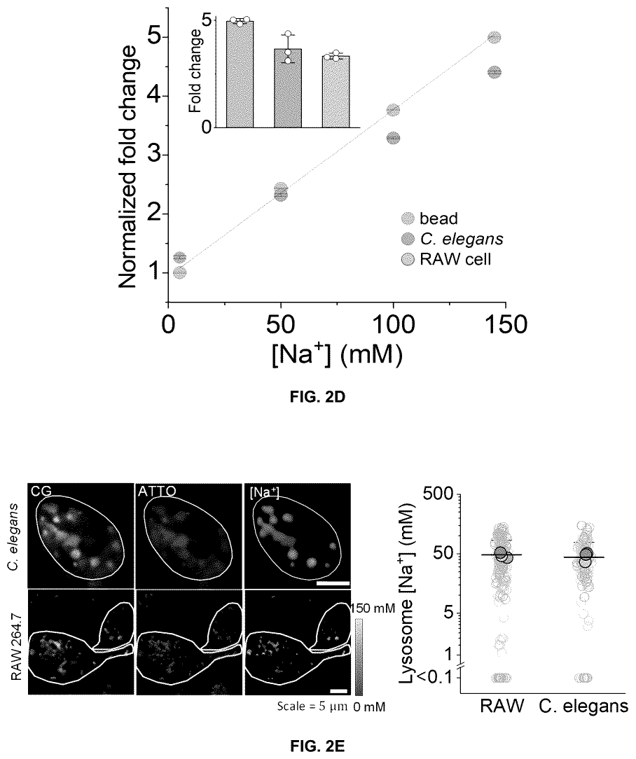

FIGS. 2A-2E illustrate in cell and in vivo calibration of RatiNa to measure lysosomal Na⁺. FIG. 2A shows fluorescence images (left) show RatiNa^{AT} (DNA, magenta) colocalized with lysosome markers (cyan), LMP-1 in C. elegans and TMR-dextran (Dex) in RAW macrophages. Percentage colocalization of RatiNa^{AT} in lysosomes (right). FIG. 2B shows ratiometric images of RatiNa^{biotin} on beads (upper panels) and RatiNa-labeled, Na⁺ clamped C. elegans lysosomes (lower panels) at the indicated Na⁺ levels. Cell outline shown in white. FIG. 2C shows G/R values of ˜80 beads (left) and ˜50 worm (right) lysosomes increase with increasing Na⁺. FIG. 2D shows linear fits of normalized G/R values of RatiNa in C. elegans lysosomes and on beads as a function of [Na⁺]. Inset shows comparable fold change in RatiNa response, from 5 to 145 mM Na⁺ and three different trials, on beads (grey), in lysosomes of RAW 264.7 macrophages in culture (cyan) and in vivo in C. elegans (orange). FIG. 2E shows absolute Na⁺ heatmaps of single, native, RatiNa-labeled lysosomes in C. elegans and RAW macrophages (n˜120 lysosomes) imaged in the CG (cyan) and ATTO647 (magenta) channels. Experiments were performed in triplicate and data from each trial is colour coded. Mean value of each trial is in filled circle of the corresponding colour⁵⁸. Error bars correspond to standard error of the mean of three trials (s.e.m.).

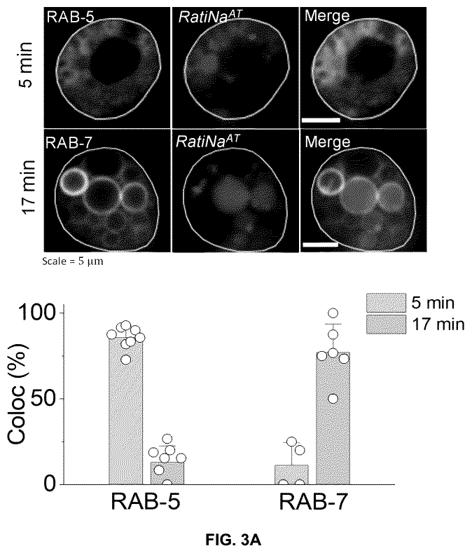

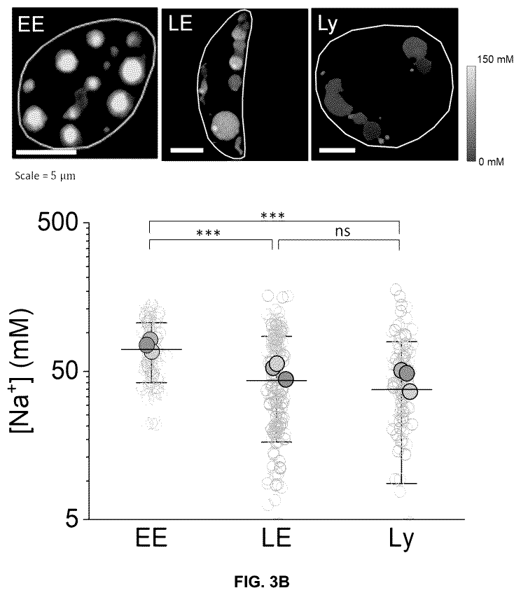

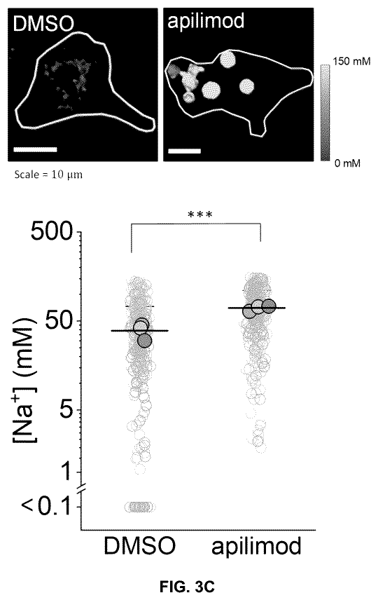

FIGS. 3A-3C illustrate that RatiNa captures physiological changes in organellar Na⁺. FIG. 3A shows RatiNa^{AT} (DNA, magenta) colocalizes with early endosome marker, RAB-5-GFP (cyan), and late endosome marker, RAB-7-GFP (cyan), in a time-dependent manner in C. elegans coelomocytes. FIG. 3B shows RatiNa maps lumenal Na⁺ levels at each stage of endosomal maturation in coelomocytes of N2 C. elegans (n˜130 endosomes per stage). Na⁺ level is highest in early endosomes (EE) and falls as EE matures into late endosomes (LE) and lysosomes (Ly). FIG. 3C shows RatiNa maps lumenal Na⁺ in lysosomes of RAW264.7 macrophages and upon inhibiting lysosomal Na⁺ channel, TPC2, with apilimod. (n˜250 lysosomes). All experiments were performed in triplicate. Data from each trial are colour coded. Mean value of each trial is in filled circle of the corresponding colour⁵⁸. *P≤0.05; **P≤0.01; ***P≤0.001 by paired t-test.

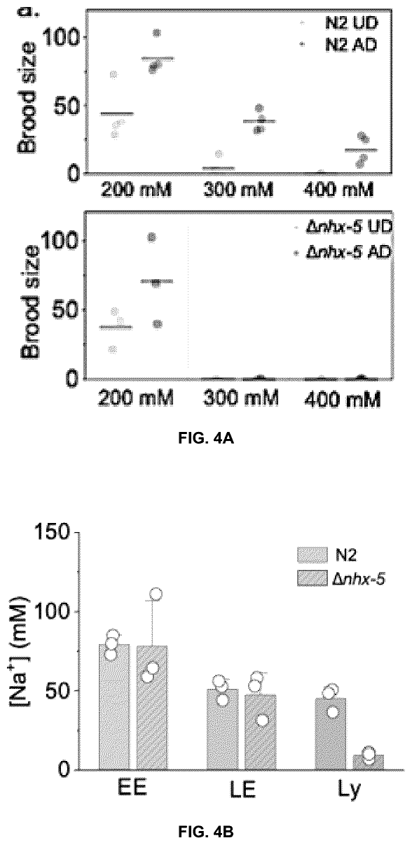

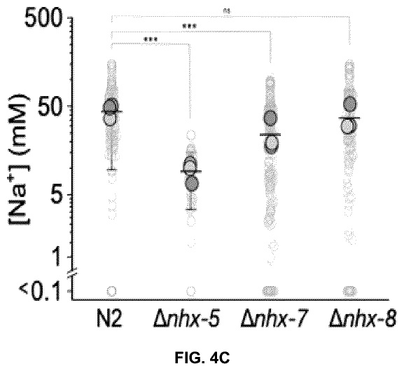

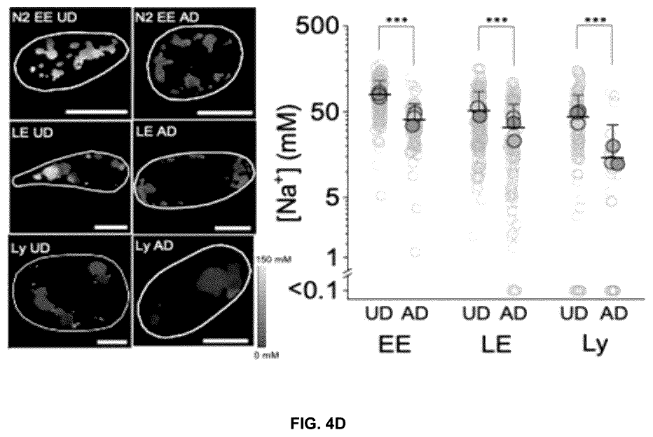

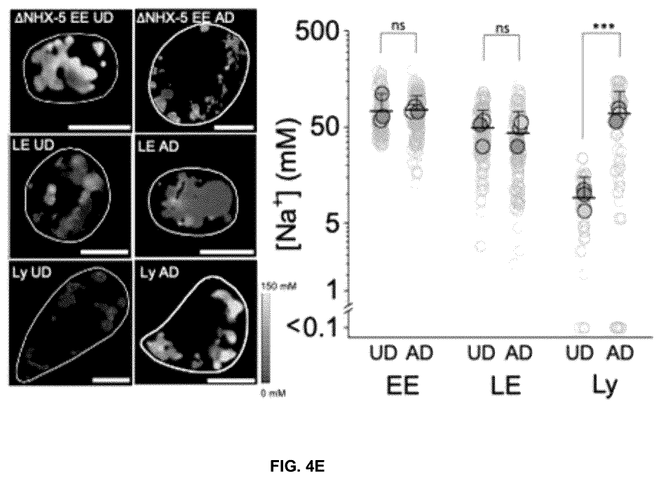

FIGS. 4A-4E illustrate that lysosomal Na⁺ transport is vital for salt adaptation in C. elegans. FIG. 4A shows brood size of unadapted (UD) and adapted (AD) N2 (top) and Δnhx-5 (bottom) worms upon exposure to progressively higher salt stress. FIG. 4B shows lumenal Na⁺ levels at each stage of the endolysosomal pathway in N2 and Δnhx-5 worms. Unlike early endosomes (EE) and late endosomes (LE), Na⁺ levels in lysosomes (Ly) of Δnhx-5 worms were significantly lower than those in N2 worms. FIG. 4C shows lumenal Na⁺ levels in single lysosomes in worms of different genetic background. Na⁺ levels were only altered in NHX-5 mutant worms. FIGS. 4D-4E show single endosome analysis of lumenal Na⁺ levels at each stage of the endolysosomal pathway in unadapted (UD) and adapted (AD) N2 (FIG. 4D) and Δnhx-5 (FIG. 4E) worms. Cell is outlined in white. All experiments were performed in triplicate and data from each trial is colour coded. Mean value of each trial is in filled circle of the corresponding colour⁵⁸. *P≤0.05; **P≤0.01; ***P≤0.001; ns, no statistical significance by paired t-test.

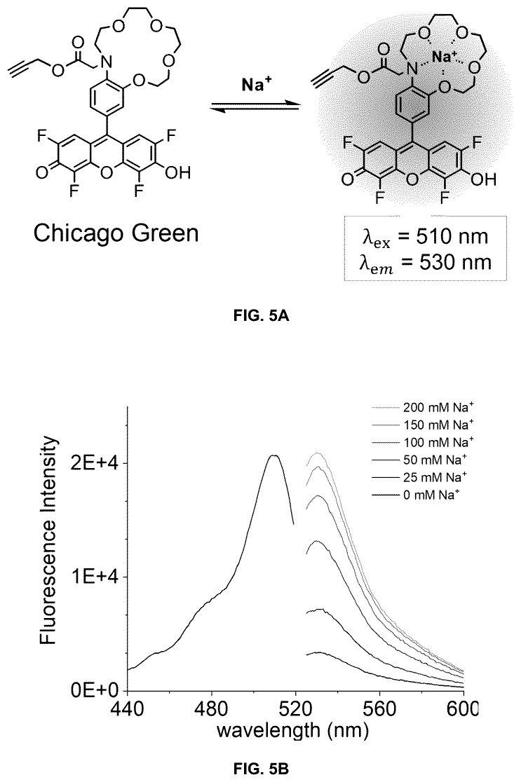

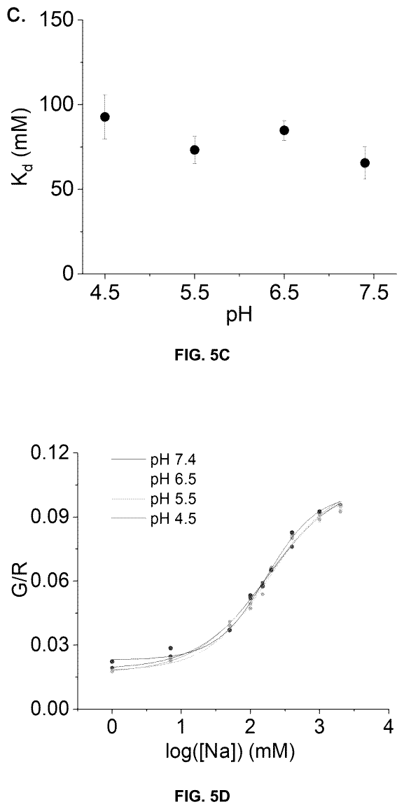

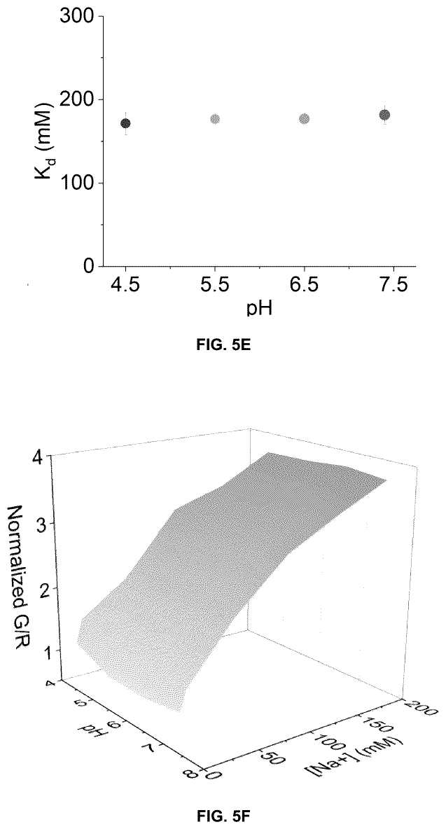

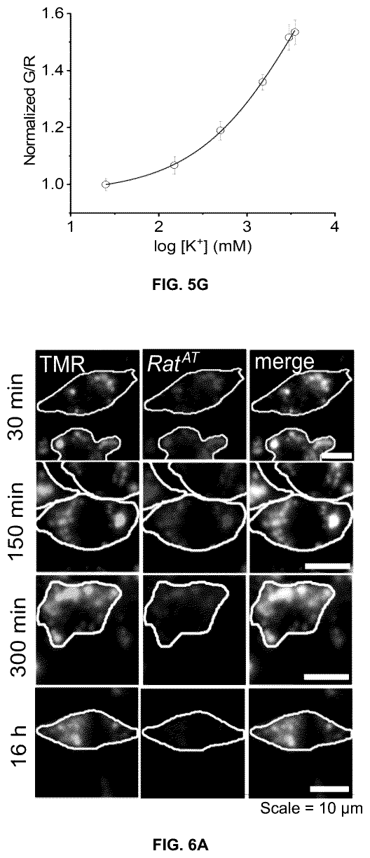

FIGS. 5A-5G illustrate that Chicago Green (CG) is pH insensitive and selective to Na⁺ prior to incorporation into RatiNa. FIG. 5A shows Na⁺ sensing mechanism of CG. FIG. 5B shows excitation (black) and emission (green) spectra of free CG increases with increasing Na⁺. FIG. 5C shows dissociation constant (K_{d}) of CG for Na⁺ does not vary with pH from pH 4.5-7.4. FIG. 5D shows individual in vitro calibration profiles of RatiNa at different pH in FIG. 1D. FIG. 5E shows K_{d} of RatiNa for Na⁺ at different pH values as calculated from FIG. 5D. K_{d} of RatiNa is higher than that of CG but is still pH invariant from pH 4.5-7.4. FIG. 5F shows magnified view of RatiNa Na⁺ calibration profile from 1 mM to 200 mM Na⁺. FIG. 5G shows RatiNa response to K⁺ yields a K_{d} of 4.5 M and 27-fold selectivity for Na⁺ over K⁺.

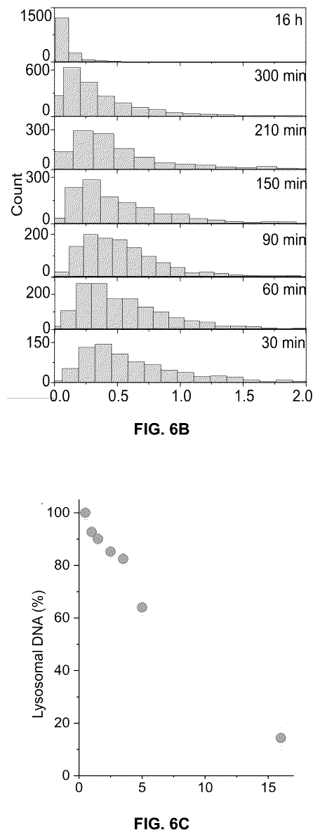

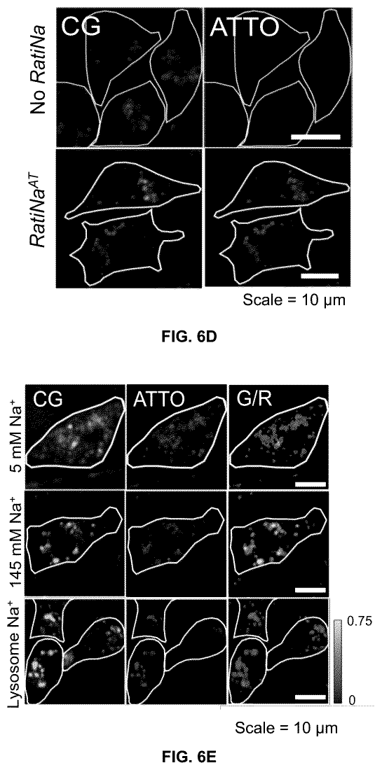

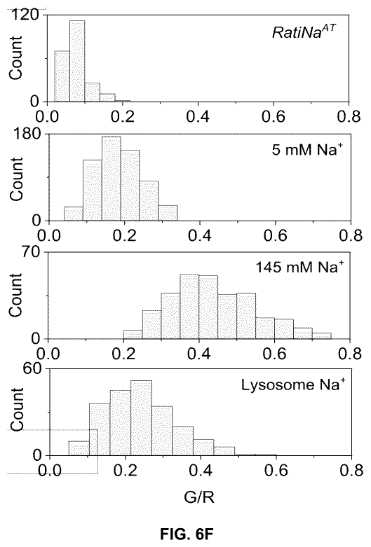

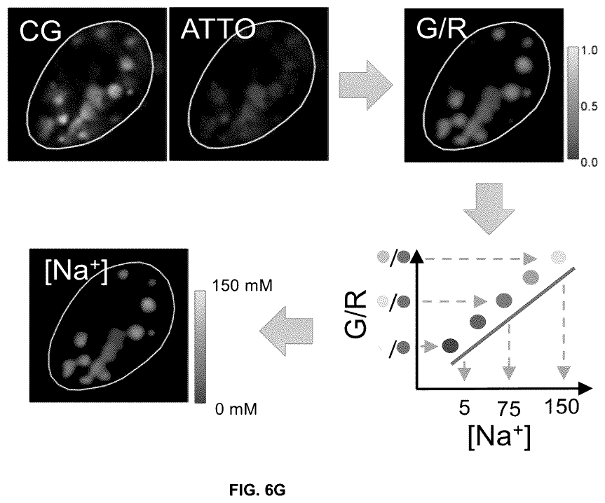

FIGS. 6A-6G illustrate that calibration of RatiNa and its stability in lysosomes of RAW264.7 macrophages. FIG. 6A shows lysosomes in RAW264.7 macrophages labeled with TMR-dextran and RatiNa^{AT}, imaged at different chase times. FIG. 6B shows single lysosome R/G analysis shows RatiNa^{AT} is stable up to 3 h. FIG. 6C shows DNA degradation as a function of chase time. Note that for 30 min chase time DNA is intact and ratiometry is valid. Error bar represents standard deviation. FIG. 6D shows fluorescence images of RatiNa^{AT} labeled RAW264.7 macrophages in CG (G) and ATTO (R) channels. FIG. 6E shows images of RatiNa-labeled lysosomes clamped at various indicated Na⁺ and in native lysosomes. G/R heat maps show adequate change that Na⁺ can be measured in native lysosomes. FIG. 6F shows distribution of G/R values of RatiNa-labeled single lysosomes. Accounting for autofluorescence, the fold change in G/R signal of RatiNa in lysosomes of RAW264.7 macrophages is comparable to that in C. elegans and on beads. FIG. 6G is a schematic of workflow from raw images to Na⁺ heat maps of single organelles. Fluorescent images in the CG (G) and ATTO (R) channels are used to construct the G/R image the Nat heatmap was generated from the calibration curve.

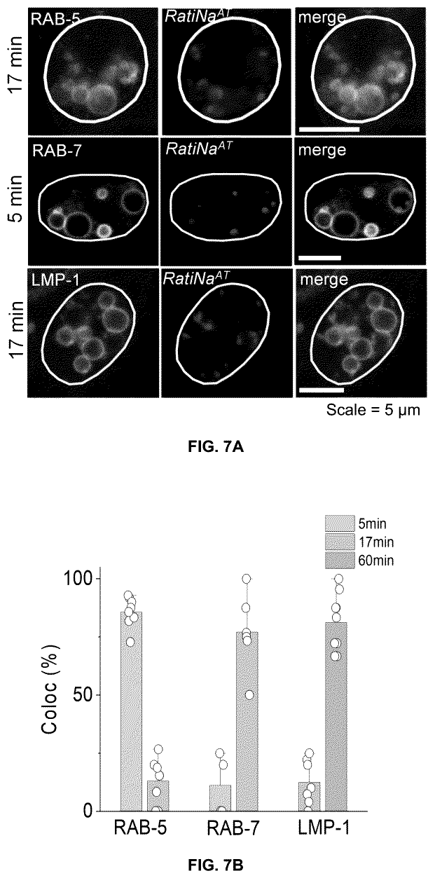

FIGS. 7A-7B illustrate specificity of RatiNa targeting to endocytic organelles. FIG. 7A shows representative images of C. elegans coelomocytes reveal negligible off-target labeling between RatiNa^{AT} and indicated endocytic markers and chase times. FIG. 7B shows quantification of data in FIG. 7A.

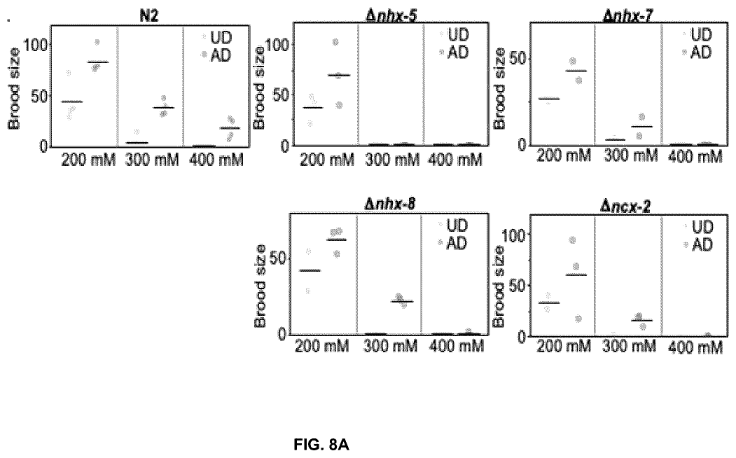

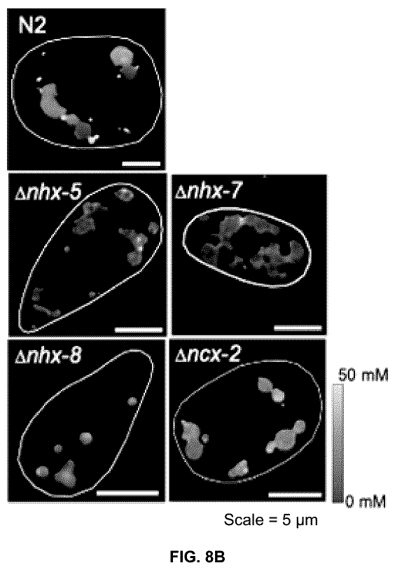

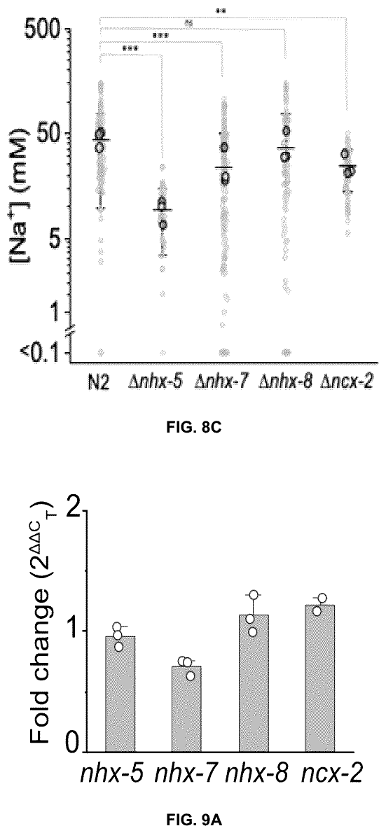

FIGS. 8A-8C illustrate that Na⁺ transporter mutant worms adapt differently to salt stress. FIG. 8A shows brood sizes of Na⁺ transporter mutants upon salt stress. FIG. 8B shows Na⁺ heatmaps of RatiNa-labeled lysosomes in wildtype (N2) worms and various Na⁺ transporter deletion mutants. FIG. 8C shows Na⁺ levels of single lysosomes of coelomocytes in N2 worms and the indicated mutants. Average Na⁺ levels in N2 worms (˜40 mM, n=120 lysosomes), Δnhx-5 (˜8 mM, n=50), Δnhx-7 (˜20 mM, n=170), Δnhx-8 (35 mM, n=120), and Δncx-2 (25 mM, n=60) are obtained from three trials (colour-coded filled circles). *P≤0.05; **P≤0.01; ***P≤0.001; ns, no statistical significance by paired t-test.

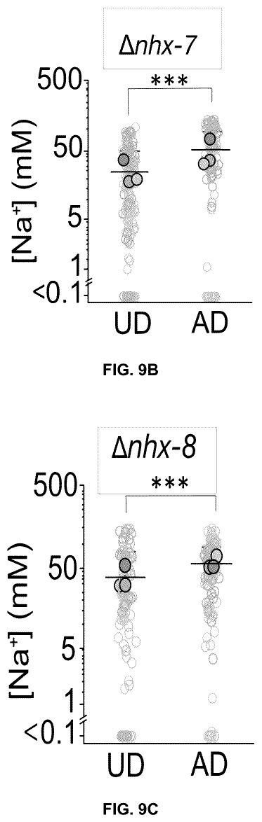

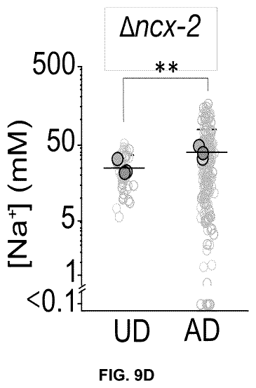

FIGS. 9A-9D illustrate that lysosomal Na⁺ levels in various Na⁺ transporter mutants increase upon salt adaptation. FIG. 9A shows that qRT-PCR shows that mRNA expression level of Na⁺ transporters do not change appreciably upon adaptation in N2 worms. Circle represents computed fold change of the indicated mRNA level between adapted and unadapted N2 worms and act-1 was used as reference gene. FIGS. 9B-9D show lysosomal Na⁺ levels of unadapted and adapted mutants, compared to unadapted N2 worms. All adapted NHX mutants worms have higher lysosomal Na⁺ compared to their unadapted counterparts. Error bar represents standard deviation. *P≤0.05; **P≤0.01; ***P≤0.001; ns, no statistical significance by paired t-test.

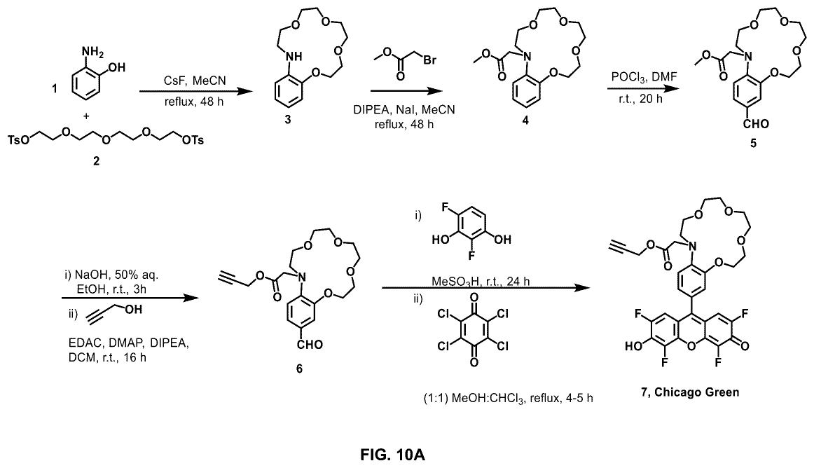

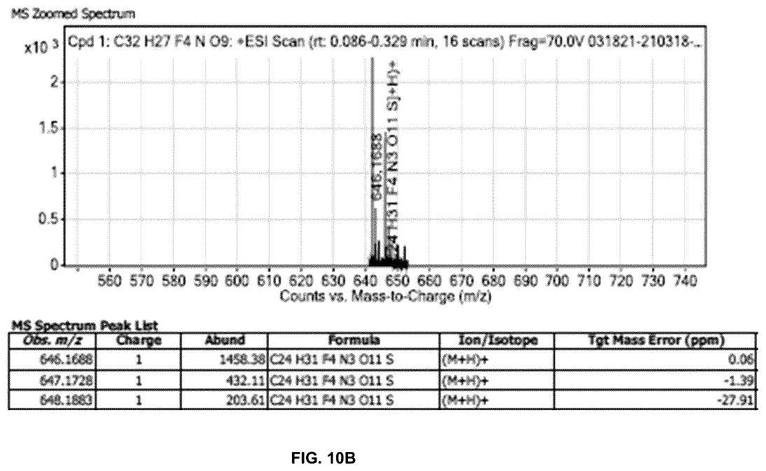

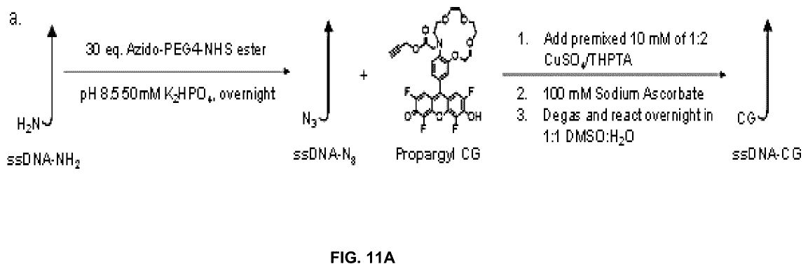

FIGS. 10A-10B illustrate the synthesis of Chicago Green. FIG. 10A shows the synthesis scheme of Chicago Green dye. FIG. 10B shows high Resolution Mass Spectrometry analysis for Chicago Green dye. m/z is identical to protonated Chicago Green dye.

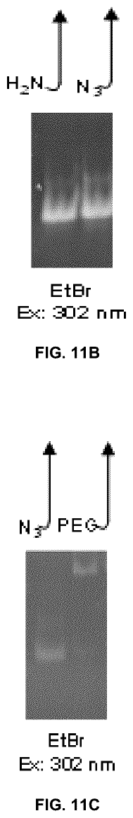

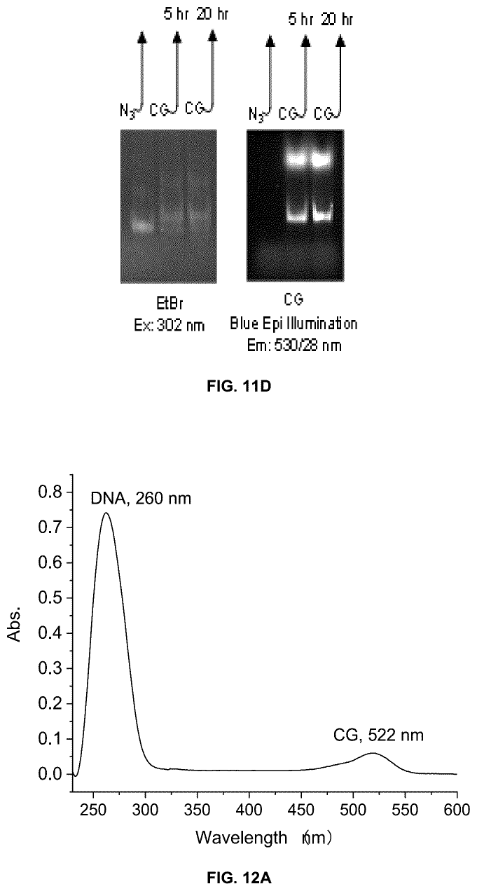

FIGS. 11A-11D illustrate the conjugation of CG to DNA. FIG. 11A is a reaction scheme of CuAAC coupling of CG to ssDNA. FIGS. 11B-11D show native PAGE of DNA intermediates and products. Unlabeled DNA strands are visualized with EtBr staining and CG conjugated DNA strand is visualized with fluorescence.

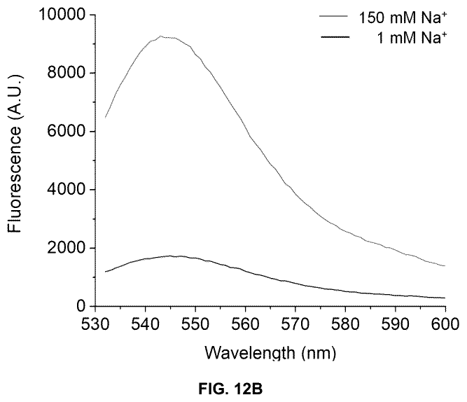

FIGS. 12A-12B illustrate the validation of CG-DNA conjugate efficiency and Na⁺ sensitivity. FIG. 12A shows a UV-Vis spectrum of ssDNA-CG. Molar ratio of DNA to CG is confirmed to be ˜ 1:1 confirming the coupling efficiency. FIG. 12B shows that ssDNA-CG fluorescence increases with high Na⁺. 1 mM and 150 mM NaCl was added to 100 nM of ssDNA-CG in pH 7.4 potassium phosphate buffer. Fluorescence maximum at 545 increases more than 5-fold.

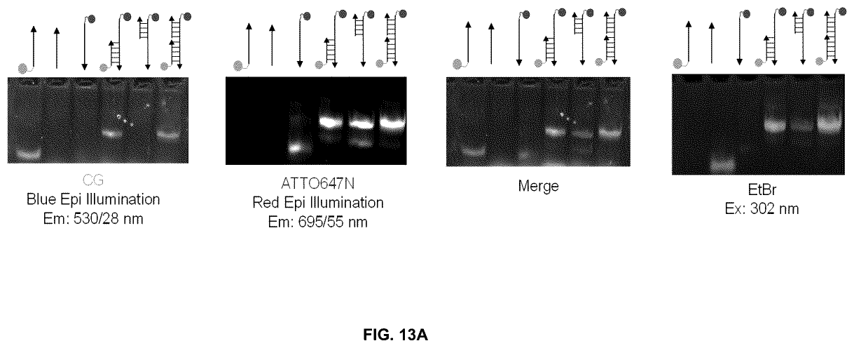

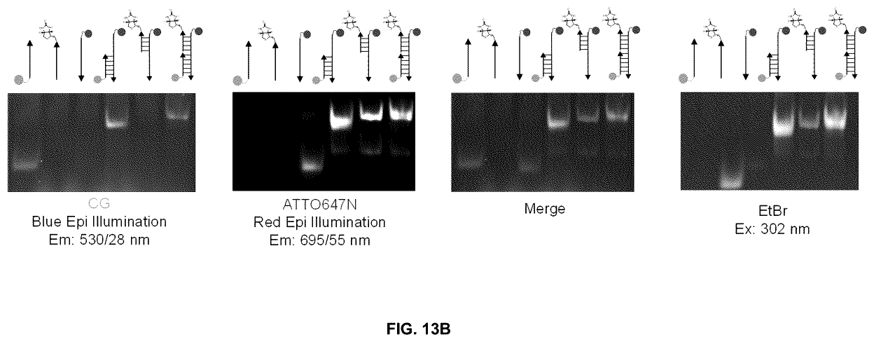

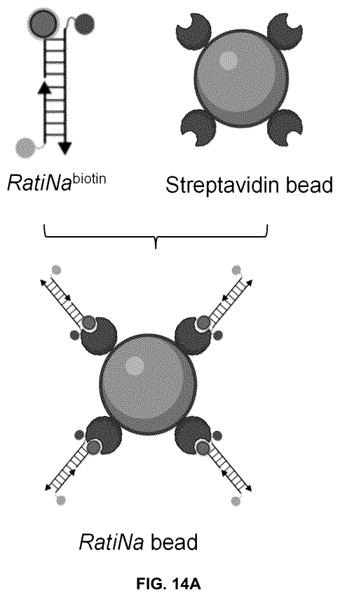

FIGS. 13A-13B illustrate the gel electrophoresis of RatiNa sensors. FIG. 13A shows 12% native PAGE of RatiNa sensor. FIG. 13B shows 12% native PAGE for biotinylated RatiNa^{biotin}. Arrowed line represents ssDNA, green circle represents CG dye and red circle represents ATTO dye.

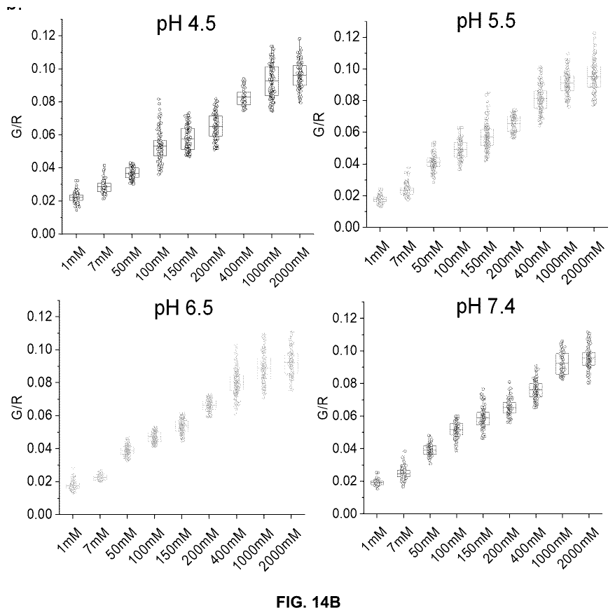

FIGS. 14A-14B illustrate the bead calibration of RatiNa sensors. FIG. 14A shows a scheme of making RatiNa bead. RatiNa^{biotin} was added to streptavidin bound bead. Beads were around the size of organelle and can be imaged in any buffer of choice for microscopy characterization. FIG. 14B shows that RatiNa bead was imaged in pH=4.5, 5.5, 6.5 and 7.4 Na⁺ calibration buffer and imaged with widefield microscope (N>100 beads, median line was shown, box represents 25% and 75% percentile, error bar represents 95% and 5% percentile).

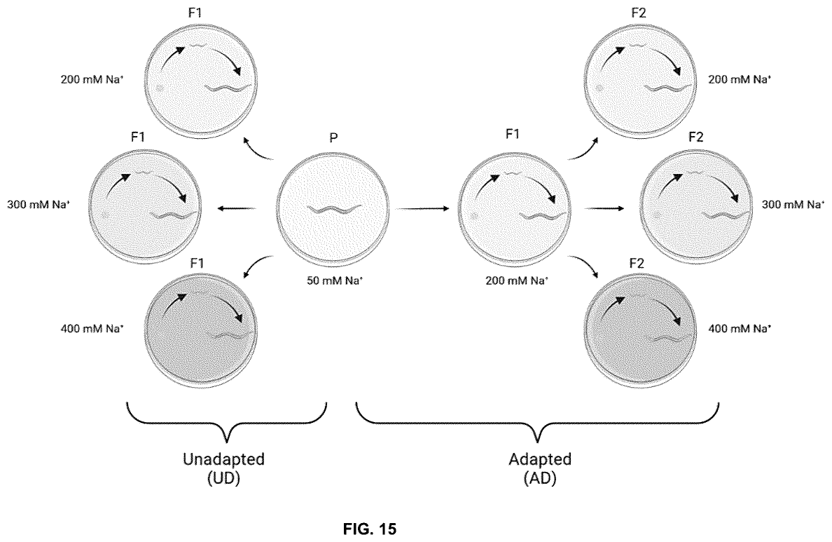

FIG. 15 illustrates a workflow of brood size assay for salt adaptation. Brood size assay was designed to compare salt adaptability from different genetic backgrounds. For unadapted worms (UD), worms were directly transferred to high Na⁺ plates from NGM plate and brood size were counted. For adapted worms, adaptation was done by allowing worms to grow for one generation at 200 mM Na⁺ plate. Then adapted worms were transferred to high Na⁺ plate and brood size were counted.

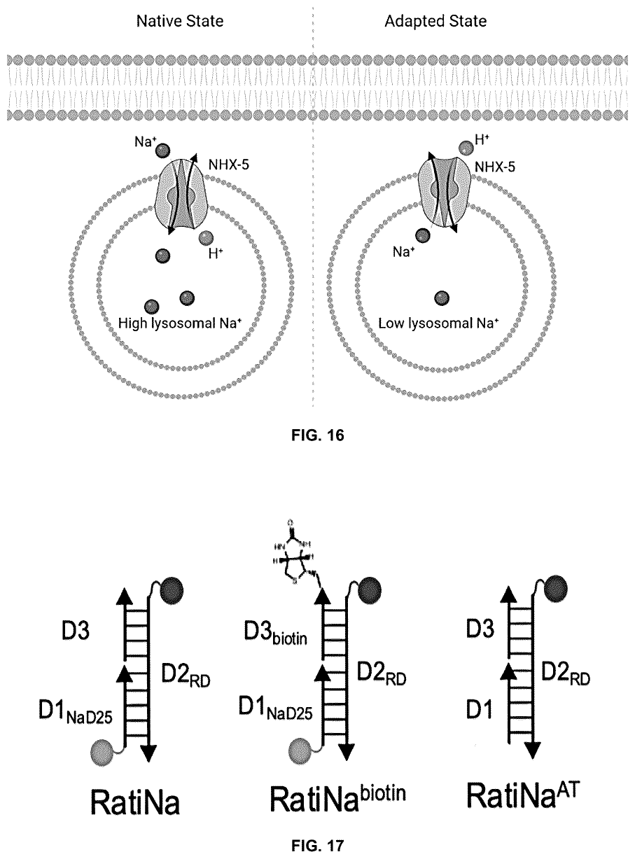

FIG. 16 illustrates a model of NHX-5 switching direction of Na⁺ transport during salt adaptation. During normal physiology conditions, NHX-5 performs as a lysosomal Na⁺ importer which helps establish normal lysosomal Na⁺ of ˜40 mM. In adapted worms NHX-5 switch direction of Na⁺ transport to exporting therefore lysosomal Na⁺ in adapted worms becomes lower. Without NHX-5 worms cannot adapt to Na⁺ properly and cannot survive at 300 mM of Na⁺.

FIG. 17 illustrates a design of RatiNa sensors. Design of sensors according to an example embodiment.

### Definitions

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used in the specification and the appended claims, the singular forms “a,” “an” and “the” include plural referents unless the context clearly dictates otherwise.

As used herein, a “fluorescent dye” or a “fluorophore” is a chemical group that can be excited by light to emit fluorescence. Some fluorophores may be excited by light to emit phosphorescence. Dyes may include acceptor dyes that are capable of quenching a fluorescent signal from a fluorescent donor dye.

As used herein, “crosslinked” refers to a covalent connection between the nucleic acid molecule and another moiety of interest, such as the Ca²⁺ fluorophore or the CI-fluorophore. In certain embodiments, the crosslink between the nucleic acid molecule and this moiety is water compatible. In certain embodiments, the crosslink between the nucleic acid molecule and this moiety is stable under physiological conditions.

As used herein, “nucleic acid,” “nucleotide sequence,” or “nucleic acid sequence” refer to a nucleotide, oligonucleotide, polynucleotide, or any fragment thereof and to naturally occurring or synthetic molecules. The term “peptide nucleic acid” or “PNA” as used herein generally refers to nucleic acid analogue in which the sugar phosphate backbone of natural nucleic acid has been replaced by a synthetic peptide backbone. The term “RNA equivalent” in reference to a DNA sequence, is composed of the same linear sequence of nucleotides as the reference DNA sequence with the exception that all occurrences of the nitrogenous base thymine are replaced with uracil, and the sugar backbone is composed of ribose instead of deoxyribose. It is understood to be a molecule that has a sequence of bases on a backbone comprised mainly of identical monomer units at defined intervals. The bases are arranged on the backbone in such a way that they can enter into a bond with a nucleic acid having a sequence of bases that are complementary to the bases of the oligonucleotide. The most common oligonucleotides have a backbone of sugar phosphate units. A distinction may be made between oligodeoxyribonucleotides, which do not have a hydroxyl group at the 2′ position, and oligoribonucleotides, which have a hydroxyl group in this position. Oligonucleotides also may include derivatives, in which the hydrogen of the hydroxyl group is replaced with organic groups, e.g., an allyl group. An oligonucleotide is a nucleic acid that includes at least two nucleotides.

One nucleic acid sequence may be “complementary” to a second nucleic acid sequence. As used herein, the terms “complementary” or “complementarity,” when used in reference to nucleic acids (i.e., a sequence of nucleotides such as an oligonucleotide or a target nucleic acid), refer to sequences that are related by base-pairing rules. For natural bases, the base pairing rules are those developed by Watson and Crick. As an example, for the sequence “T-G-A”, the complementary sequence is “A-C-T.” Complementarity can be “partial,” in which only some of the bases of the nucleic acids are matched according to the base pairing rules. Alternatively, there can be “complete” or “total” complementarity between the nucleic acids. The degree of complementarity between the nucleic acid strands has effects on the efficiency and strength of hybridization between the nucleic acid strands.

Oligonucleotides as described herein may be capable of forming hydrogen bonds with oligonucleotides having a complementary base sequence. These bases may include the natural bases such as A, G, C, T and U, as well as artificial bases. An oligonucleotide may include nucleotide substitutions. For example, an artificial or modified base may be used in place of a natural base such that the artificial base exhibits a specific interaction that is similar to the natural base.

An oligonucleotide that is complementary to another nucleic acid will “hybridize” to the nucleic acid under suitable conditions (described below). As used herein, “hybridization” or “hybridizing” refers to the process by which an oligonucleotide single strand anneals with a complementary strand through base pairing under defined hybridization conditions. “Specific hybridization” is an indication that two nucleic acid sequences share a high degree of complementarity. Specific hybridization complexes form under permissive annealing conditions and remain hybridized after any subsequent washing steps. “Hybridizing” sequences which bind under conditions of low stringency are those which bind under non-stringent conditions (6×SSC/50% formamide at room temperature) and remain bound when washed under conditions of low stringency (2×SSC, 42° C.). Hybridizing under high stringency refers to the above conditions in which washing is performed at 2×SSC, 65° C. (where SSC is 0.15M NaCl, 0.015M sodium citrate, pH 7.2).

In view of the present disclosure, the methods described herein can be configured by the person of ordinary skill in the art to meet the desired need.

The oligonucleotides and nucleic acid molecules in the compositions and methods described herein may include one or more labels. Nucleic acid molecules can be labeled by incorporating moieties detectable by one or more means including, but not limited to, spectroscopic, photochemical, biochemical, immunochemical, or chemical assays. The method of linking or conjugating the label to the nucleotide or oligonucleotide depends on the type of label(s) used and the position of the label on the nucleotide or oligonucleotide.

As used herein, “labels” are chemical or biochemical moieties useful for labeling a nucleic acid. “Labels” include, for example, fluorescent agents, chemiluminescent agents, chromogenic agents, quenching agents, radionucleotides, enzymes, substrates, cofactors, inhibitors, nanoparticles, magnetic particles, and other moieties known in the art. Labels are capable of generating a measurable signal and may be covalently or noncovalently joined to an oligonucleotide or nucleotide.

In some embodiments, the nucleic acid molecules may be labeled with a “fluorescent dye” or a “fluorophore.” Dyes that may be used in the disclosed methods include, but are not limited to, the following dyes sold under the following trade names: 1,5 IAEDANS; 1,8-ANS; 4-Methylumbelliferone; 5-carboxy-2,7-dichlorofluorescein; 5-Carboxyfluorescein (5-FAM); 5-Carboxytetramethylrhodamine (5-TAMRA); 5-Hydroxy Tryptamine (HAT); 5-ROX (carboxy-X-rhodamine); 6-Carboxyrhodamine 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633 ™; Alexa Fluor 647™; Alexa Fluor 660™; Alexa Fluor 680™; Alizarin Complexon; Alizarin Red; Allophycocyanin (APC); AMC; AMCA-S; AMCA (Aminomethylcoumarin); AMCA-X; Aminoactinomycin D; Aminocoumarin; Aminomethylcoumarin (AMCA); Anilin Blue; Anthrocyl stearate; APC (Allophycocyanin); APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); Berberine Sulphate; Beta Lactamase; BFP blue shifted GFP (Y66H); Blue Fluorescent Protein; BFP/GFP FRET; Bimane; Bisbenzamide; Bisbenzimide (Hoechst); Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy FL; Bodipy FL ATP; Bodipy FI-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Orange; Calcofluor White; Cascade Blue™; Cascade Yellow; Catecholamine; CCF2 (GeneBlazer); CFDA; CFP-Cyan Fluorescent Protein; CFP/YFP FRET; Chlorophyll; Chromomycin A; CL-NERF (Ratio Dye, pH); CMFDA; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM Methylcoumarin; CTC; CTC Formazan; Cy2™; Cy3. 18; Cy3.5™; Cy3™; Cy5.18; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); Dichlorodihydrofluorescein Diacetate (DCFH); DiD-Lipophilic Tracer; DiD (DiIC18 (5)); DIDS; Dihydorhodamine 123 (DHR); DiI (DiIC18 (3)); Dinitrophenol; DiO (DiOC18 (3)); DIR; DIR (DiIC18 (7)); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (III) chloride; EYFP; Fast Blue; FDA; Feulgen (Pararosaniline); Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™; Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer (CCF2); GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1; Indodicarbocyanine (DiD); Indotricarbocyanine (DiR); Intrawhite Cf; JC-1; JO-JO-1; JO-PRO-1; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; NED™; Nitrobenzoxadidole; Noradrenaline; Nuclear Fast Red; Nuclear Yellow; Nylosan Brilliant lavin E8G; Oregon Green; Oregon Green 488-X; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed [Red 613]; Phloxin B (Magdala Red); Phorwite AR; Phorwite BKL; Phorwite Rev; Phorwite RPA; Phosphine 3R; Phycoerythrin B [PE]; Phycoerythrin R [PE]; PKH26 (Sigma); PKH67; PMIA; Pontochrome Blue Black; POPO-1; POPO-3; PO-PRO-1; PO-PRO-3; Primuline; Procion Yellow; Propidium lodid (PI); PYMPO; Pyrene; Pyronine; Pyronine B; Pyrozal Brilliant Flavin 7GF; QSY 7; Quinacrine Mustard; Red 613 [PE-TexasRed]; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycocyanine; R-phycoerythrin (PE); RsGFP; S65A; S65C; S65L; S65T; Sapphire GFP; SBFI; Serotonin; Sevron Brilliant Red 2B; Sevron Brilliant Red 4G; Sevron Brilliant Red B; Sevron Orange; Sevron Yellow L; sgBFPT; sgBFP™ (super glow BFP); sgGFP™; sgGFP™ (super glow GFP); SITS; SITS (Primuline); SITS (Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3-sulfopropyl) quinolinium); Stilbene; Sulphorhodamine B can C; Sulphorhodamine G Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; TETT; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC TetramethylRodaminelsoThioCyanate; True Blue; TruRed; Ultralite; Uranine B; Uvitex SFC; VIC®; wt GFP; WW 781; X-Rhodamine; XRITC; Xylene Orange; Y66F; Y66H; Y66W; Yellow GFP; YFP; YO-PRO-1; YO-PRO-3; YOYO-1; YOYO-3; pHrodo™ (available from Thermo Fischer Scientific, Inc. Waltham, MA), and salts thereof.

Fluorescent dyes or fluorophores may include derivatives that have been modified to facilitate conjugation to another reactive molecule. As such, fluorescent dyes or fluorophores may include amine-reactive derivatives such as isothiocyanate derivatives and/or succinimidyl ester derivatives of the fluorophore.

The nucleic acid molecules of the disclosed compositions and methods may be labeled with a quencher. Quenching may include dynamic quenching (e.g., by FRET), static quenching, or both. Illustrative quenchers may include Dabcyl. Illustrative quenchers may also include dark quenchers, which may include black hole quenchers sold under the tradename “BHQ” (e.g., BHQ-0, BHQ-1, BHQ-2, and BHQ-3, Biosearch Technologies, Novato, Calif.). Dark quenchers also may include quenchers sold under the tradename “QXL™” (Anaspec, San Jose, Calif.). Dark quenchers also may include DNP-type non-fluorophores that include a 2,4-dinitrophenyl group.

The labels can be conjugated to the nucleic acid molecules directly or indirectly by a variety of techniques. Depending upon the precise type of label used, the label can be located at the 5′ or 3′ end of the oligonucleotide, located internally in the oligonucleotide's nucleotide sequence, or attached to spacer arms extending from the oligonucleotide and having various sizes and compositions to facilitate signal interactions. Using commercially available phosphoramidite reagents, one can produce nucleic acid molecules containing functional groups (e.g., thiols or primary amines) at either terminus, for example by the coupling of a phosphoramidite dye to the 5′ hydroxyl of the 5′ base by the formation of a phosphate bond, or internally, via an appropriately protected phosphoramidite.

Nucleic acid molecules may also incorporate functionalizing reagents having one or more sulfhydryl, amino or hydroxyl moieties into the nucleic acid sequence. For example, a 5′ phosphate group can be incorporated as a radioisotope by using polynucleotide kinase and [γ32P] ATP to provide a reporter group. Biotin can be added to the 5′ end by reacting an aminothymidine residue, introduced during synthesis, with an N-hydroxysuccinimide ester of biotin. Labels at the 3′ terminus, for example, can employ polynucleotide terminal transferase to add the desired moiety, such as for example, cordycepin, 35S-dATP, and biotinylated dUTP.

Oligonucleotide derivatives are also available as labels. For example, etheno-dA and etheno-A are known fluorescent adenine nucleotides which can be incorporated into a reporter. Similarly, etheno-dC is another analog that can be used in reporter synthesis. The reporters containing such nucleotide derivatives can be hydrolyzed to release much more strongly fluorescent mononucleotides by the polymerase's 5′ to 3′ nuclease activity as nucleic acid polymerase extends a primer during PCR.

The current methods, nucleic acids, and nucleic acid complexes may be used in combination with additional nucleic acid based sensors, such as those described in WO 2015/159122, which is herein incorporated by reference.

In the present disclosure, PNA or PNA strand or PNA sequence is used interchangeably and has the same scope or meaning. In the present disclosure, DNA or DNA strand or DNA sequence is used interchangeably and has the same scope or meaning. In the present disclosure, RNA or RNA strand or RNA sequence is used interchangeably and has the same scope or meaning.

In some embodiments, the nucleic acid complex of the present disclosure self assembles two or all three strands through Watson-Crick base pairing, which is stable under physiological conditions.

In embodiments of the present disclosure, two types of targeting moiety may be used: A) DNA only and B) a combination of DNA and RNA. The targeting moiety comprising only DNA hybridizes to normalizing module to form the dsDNA domain required for intracellular targeting via an anionic ligand binding receptor (ALBR). The RNA sequence used in combination with DNA in the targeting moiety is used to achieve targeting to Transferrin pathway.

In some embodiments, a DNA strand is used as first strand and/or the second strand. In an embodiment of the present disclosure, the nucleic acid complex has a dsDNA part (minimum 15 bp sequence) resulting from the hybridization of the first strand and the second strand, or the first strand and the third strand, or the first strand with the second strand and the third strand.

### Detection of Nat Concentration in Samples

The methods described herein may be used to monitor the sodium concentration in real-time during cellular processes. In some embodiments, the methods are for monitoring endocytosis. While not wishing to be limited by theory, acidification plays a major role in facilitating cargo dissociation from receptors or in mediating cellular entry of toxins and viruses during endocytosis. In certain embodiments, the nucleic acid complex exhibits a pH response inside cells illustrated by the capture of spatiotemporal pH changes associated with endocytosis in living cells.

Fluorescence in the sample can be measured in a variety of ways, such as using a fluorometer or fluorescence microscopy. In general, excitation radiation, from an excitation source having a first wavelength, passes through excitation optics. The excitation optics cause the excitation radiation to excite the sample. In response, labels in the sample emit radiation which has a wavelength that is different from the excitation wavelength. The device can include a temperature controller to maintain the sample at a specific temperature while it is being scanned. If desired, a multi-axis translation stage can be used to move a microtiter plate holding a plurality of samples in order to position different wells to be exposed. The multi-axis translation stage, temperature controller, auto-focusing feature, and electronics associated with imaging and data collection can be managed by an appropriately programmed digital computer. The computer also can transform the data collected during the assay into another format for presentation.

In some embodiments, the detecting includes measuring the magnitude of the signal generated, wherein the magnitude indicates the sodium concentration in the cell or region thereof. As used herein, an “increase” (or “decrease”) in a signal from the nucleic acid complex refers to the change in a signal in the sample compared to a reference sample. The reference sample may be a control sample (e.g., an untreated population of cells where the effects of a drug or agent are being examined), or it may be the same sample at a different period of time, for instance, where the sodium concentration is being monitored to follow one or more cellular processes. In other embodiments, the reference sample is the fluorescence intensity of a reference fluorophore, for example, a reference fluorophore linked to the nucleic acid as otherwise described herein.

As one of skill in the art will understand, there will be a certain degree of uncertainty involved in making this determination. Therefore, the standard deviations of the control group levels can be used to make a probabilistic determination and the method of this disclosure are applicable over a wide range of probability-based determinations. Thus, for example, and not by way of limitation, in one embodiment, if the measured level of signal falls within 2.5 standard deviations of the mean of any of the control groups, then that sample may be assigned to that group. In another embodiment if the measured level of signal falls within 2.0 standard deviations of the mean of any of the control groups then that sample may be assigned to that group. In still another embodiment, if the measured level of signal falls within 1.5 standard deviations of the mean of any of the control groups then that sample may be assigned to that group. In yet another embodiment, if the measured level of signal is 1.0 or less standard deviations of the mean of any of the control groups levels then that sample may be assigned to that group. Thus, this process allows determination, with various degrees of probability, in which group a specific sample should be placed.

Statistical methods can also be used to set thresholds for determining when the signal intensity in a test sample can be considered to be different than or similar to the reference level. In addition, statistics can be used to determine the validity of the difference or similarity observed between a test sample's signal intensity and the reference level. Useful statistical analysis methods are described in L. D. Fisher & G. vanBelle, Biostatistics: A Methodology for the Health Sciences (Wiley-Interscience, NY, 1993). For instance, confidence (“p”) values can be calculated using an unpaired 2-tailed t test, with a difference between groups deemed significant if the p value is less than or equal to 0.05.

To minimize artefactually low fluorescence measurements that occur due to cell movement or focusing, the fluorescence of the nucleic acid complex can be compared to the fluorescence of a second sensor, e.g., a second nucleic acid complex that is also present in the measured sample, or a reference fluorophore. The second nucleic acid complex or reference fluorophore should have an emission spectra distinct from the first nucleic acid complex so that the emission spectra of the two sensors can be distinguished. Because experimental conditions such as focusing and cell movement will affect fluorescence of the second sensor as well as the first sensor, comparing the relative fluorescence of the two sensors or fluorophores may allow for the normalization of fluorescence. A convenient method of comparing the samples is to compute the ratio of the fluorescence of the first fluorophore to that of the second reference fluorophore. Accordingly, in certain embodiments as otherwise described herein, the method further comprises measuring the intensity of the reference fluorophore, and normalizing the Na⁺ fluorescence to the reference fluorescence.

In some embodiments, the Na⁺ concentration is determined by comparing the measured signal to a reference value. In some embodiments, the Na⁺ concentration is determined by comparing the measured signal to a reference value. In some embodiments, the signal value and/or reference value is normalized. In some embodiments, the method further comprises creating a standard curve. A standard curve can be created by measuring the signal intensity at different known Na⁺ concentration values. A curve can be plotted as signal intensity vs. Na⁺ concentration. The signal intensity of an unknown Na⁺ concentration can then be determined by finding the corresponding reference value on the plot. Accordingly, in certain embodiments as otherwise described herein, the nucleic acid complex further comprises a reference fluorophore linked to the first single-stranded nucleic acid molecule or the second single-stranded nucleic acid molecule. For example, in particular embodiments, the nucleic acid complex further comprises a reference fluorophore linked to the second single-stranded nucleic acid. For example, in certain embodiments as otherwise described herein, the reference fluorophore is a fluorophore as otherwise described herein, such as an Atto dye, an Alexa Fluor® dye, a Cy® dye, or a BODIPY dye. In particular embodiments, the reference fluorophore comprises an ATTO647 fluorophore.

The present disclosure provides for the determination of wide ranges of Na⁺ concentration. Accordingly, in various embodiments as otherwise described herein, the he determined Na⁺ concentration is in a range of 10 μM to 500 mM, e.g., in the range of 100 μM to 150 mM, or in the range of 1 mM to 150 mM.

Advantageously, the reference fluorophore can be selected so that the fluorophore is relatively insensitive to both pH and Na⁺ concentration. Accordingly, in certain embodiments as otherwise described herein, the reference fluorophore is pH insensitive and Na⁺ concentration insensitive. For example, the reference fluorophore may be insensitive to pH and Na⁺ concentration within physiological ranges, for example between pH 5.5-8, and 10 μM to 500 mM Na⁺ concentration.

The present disclosure provides a Na⁺ fluorophore which may have low sensitivity to pH. This enables accurate determination of Na⁺ concentration without having to adjust for the confounding factor of any pH influence. In certain embodiments as otherwise described herein, the Na⁺ fluorophore is pH insensitive. As used herein, “pH insensitive” means that the measured Na⁺ concentration does not vary more than 10% as pH is adjusted from 4.5 to 7.4 (e.g., no more than 5%, or no more than 1%).

In some embodiments, the signal intensity changes by at least twenty percent as the Na⁺ concentration is raised. In some embodiments, the signal intensity changes by at least 10, 20, 25, 30, 35, 40, 45, 50, 55, 60, 65, 70, 75, 80, 85, or 90% or any derivable range therein when the Na⁺ concentration is raised.

The present disclosure provides for Na⁺ fluorophores that have high sensitivity to Na⁺ concentration but may present low pH sensitivity, as well as low sensitivity to other ions. In certain embodiments as otherwise described herein, the Na⁺ fluorophore comprises a 15-crown-5 ether moiety. For example, in particular embodiments, the Na⁺ fluorophore comprises a 1-aza-15-crown-5 ether moiety. In some embodiments, the Na⁺ comprises:

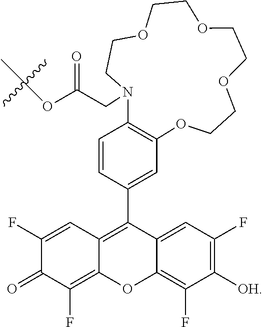 [SMILES: *OC(=O)CN1CCOCCOCCOCCOc2cc(-c3c4cc(F)c(=O)c(F)c-4oc4c(F)c(O)c(F)cc34)ccc21]

In certain embodiments, the present inventors have determined that the Na⁺ fluorophore can be linked to the first single-stranded nucleic acid molecule using a linker, e.g., a linker moiety that is stable under physiological conditions. For example, in certain embodiments, the linker can be formed using click chemistry. Thus, in certain embodiments, the Na⁺ fluorophore is linked to the first strand through a triazole, thioether, or alkenyl sulfide group. For example, the triazole, thioether, or alkenyl sulfide group can be formed from an azide or thiol moiety on the Na⁺ fluorophore and a alkyne or alkene moiety on the first single-stranded nucleic acid molecule. In another example, the triazole, thioether, or alkenyl sulfide group can be formed from an azide or thiol moiety on the first single-stranded nucleic acid molecule and a alkyne or alkene moiety on the Ca²⁺ fluorophore. In particular embodiments, the Na⁺ fluorophore comprises:

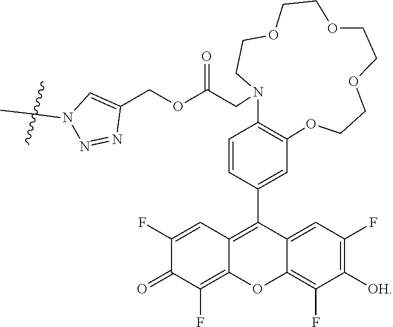 [SMILES: *n1cc(COC(=O)CN2CCOCCOCCOCCOc3cc(-c4c5cc(F)c(=O)c(F)c-5oc5c(F)c(O)c(F)cc45)ccc32)nn1]

in certain embodiments as otherwise described herein, the nucleic acid complex further comprises a third single-stranded nucleic acid molecule that is partially complementary to the second single-stranded molecule.

The nucleic acid molecules and complexes of the disclosure, in some embodiments, comprise a targeting moiety, such as a nucleic acid, small molecule, or polypeptide that has an affinity for a certain target or, by virtue of its chemical makeup, is targeted to a particular location in the cell. The targeting moiety can act as a handle to target the nucleic acid complexes of the disclosure to different subcellular locations. The targeting moiety may be a nucleic acid that binds to a receptor protein, and the receptor protein may be one that is intracellularly targeted or conjugated to a protein that is intracellularly targeted. The targeting moiety or receptor protein may be a targeting nucleic acid or a protein such as a plasma membrane protein that is endocytosable, any proteins that possess a natural receptor, a protein that traffics between intracellular locations via the plasma membrane, toxins, viruses and viral coat proteins, cell penetrating peptides, signal sequences, intracellular targeting sequences, small organic molecules, endocytic ligands and trafficking proteins. In some embodiments, the targeting moiety is an aptamer, a duplex domain targeted to an artificial protein receptor, a nucleic acid sequence that binds an anionic-ligand binding receptor, or an endocytic ligand. The targeting moiety may also be a G4 core sequence or ribozyme.

In some embodiments, the targeting moiety is a nucleic acid sequence. In some embodiments, the targeting moiety has a cognate artificial protein receptor. The artificial receptor may be, for example, a single chain variable fragment (scFv), transcription factor, Zn-fingered protein, leucine zipper, or DNA binding immunoglobulin, In some embodiments, the targeting moiety is encoded on the same nucleic acid strand as the first and/or second single-stranded nucleic acid molecule. In some embodiments, the targeting moiety is selected from an aptamer, a duplex domain targeted to an artificial protein receptor, a nucleic acid sequence that binds an anionic-ligand binding receptor, and an endocytic ligand. In some embodiments, the targeting moiety comprises a peptide directly or indirectly conjugated to the nucleic acid molecule. In some embodiments, the targeting moiety peptide comprises one or more of a fusogenic peptide, a membrane-permeabilizing peptide, a sub-cellular localization sequence, or a cell-receptor ligand. In some embodiments, the sub-cellular localization sequence targets the nucleic acid complex to a region of the cell where spatial localization of a targeted protein is present. In some embodiments, the sub-cellular localization sequence targets the nucleic acid complex to a region of the cell selected from the group consisting of: the cytosol, the endoplasmic reticulum, the mitochondrial matrix, the chloroplast lumen, the medial trans-Golgi cistemae, the lumen of lysosome, the lumen of an endosome, the peroxisome, the nucleus, and a specific spatial location on the plasma membrane. In some embodiments, the sub-cellular organelle is one that exchanges membrane directly or indirectly with the plasma membrane.

In certain embodiments as otherwise described herein, the targeting moiety is encoded on the same nucleic acid strand as the first single-stranded nucleic acid molecule, the second single-stranded nucleic acid molecule, the third single-stranded nucleic acid molecule, or any combination thereof. For example, in particular embodiments, the targeting moiety is located on the third single-stranded nucleic acid molecule.

In certain embodiments of the nucleic acid complexes of the disclosure, each of the first single-stranded nucleic acid molecule the second single-stranded nucleic acid molecule, and/or the third single-stranded nucleic acid molecule is independently less than 200 nucleotides, e.g., less than 100 nucleotides, or less than 50 nucleotides. In some embodiments, each of the first single-stranded nucleic acid molecule the second single-stranded nucleic acid molecule, and/or the third single-stranded nucleic acid molecule is independently less than, at least, or exactly 20, 30, 40, 60, 80, 100, 125, 150, 175, or 200 nucleotides in length, or any derivable range therein. In particular embodiments, the first and third single-stranded nucleic molecules together are the same length (e.g., within 2 nucleotides, or within 1 nucleotide, or exactly) as the second single-stranded nucleic acid molecule.

In certain embodiments as otherwise described herein, the first single-stranded nucleic acid molecule has the sequence 5′-CG-ATC AAC ACT GCA TAT ATA TAC GAC C-3′ [SEQ ID NO: 1] or 5′-ATC AAC ACT GCA TAT ATA TAC GAC C-3′ [SEQ ID NO:2]; and the second single-stranded nucleic acid molecule has the sequence 5′-ATTO647N—C ACT GCA CAC CAG ACA GCA A G GTC GTA TAT ATA TGC AGT GTT GAT-3′ [SEQ ID NO:3]. In certain embodiments as otherwise described herein, the nucleic acid complex further comprises a third single-stranded nucleic acid molecule that is partially complementary to the second single-stranded molecule, and wherein the third single-stranded nucleic acid molecule has the sequence 5′-T TGC TGT CTG GTG TGC AGT G-BioTEG-3′ [SEQ ID NO:4] or 5′-T TGC TGT CTG GTG TGC AGT G-3′ [SEQ ID NO:5], wherein bioTEG is a biotin-triethylene glycol moiety.

In another aspect, the present disclosure provides for a compound having the structure:

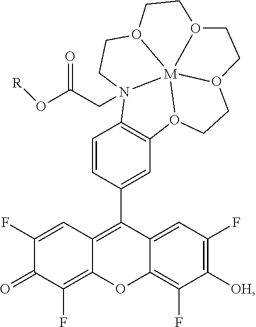

wherein:
M is an alkali metal ion, or is absent; and
R is H, C₁-C₁₂ alkyl, (C₁-C₁₂ alkyl)-SH, (C₁-C₁₂ alkyl)-N₃, C₂-C₁₂ alkenyl group, or C₂-C₁₂ alkynyl.

For example, the compound as otherwise described herein may be used as a fluorophore, such as a Na⁺ fluorophore, according to the methods as otherwise described herein, and/or incorporated into a nucleic acid complex as otherwise described herein.

In particular embodiments, M is Na⁺, or is absent. In some embodiments as otherwise described herein, R is a linker as otherwise described herein, e.g., C₂-C₆ alkynyl. In particular embodiments as otherwise described herein, R is —CH₂—C≡CH.

The methods of the disclosure, in certain embodiments, are suitable for measuring the concentration of Na⁺ in early endosome, late endosome, plasma membrane, lysosome, autophagolysosome, recycling endosome, cis Golgi network (CGN), trans Golgi network (TGN), endoplasmic reticulum (ER), peroxisomes, or secretory vesicles. In certain embodiments, the methods of the disclosure are suitable for measuring the concentration of Na⁺ in early endosome, late endosome, plasma membrane, lysosome, autophagolysosome, recycling endosome, or TGN.

In general, any sample containing Na⁺ can be used in the methods of the disclosure. In some embodiments, the sample is a biological sample selected from a cell, cell extract, cell lysate, tissue, tissue extract, bodily fluid, serum, blood and blood product. In some embodiments, the sample is a live cell. In some embodiments, the sample is a biological sample from a patient.

The nucleic acid complexes as described herein can be readily introduced into a host cell, e.g., a mammalian (optionally human), bacterial, parasite, yeast or insect cell by any method in the art. For example, nucleic acids can be transferred into a host cell by physical, chemical or biological means. It is readily understood that the introduction of the nucleic acid molecules yields a cell in which the intracellular pH may be monitored. Thus, the method can be used to measure intracellular pH in cells cultured in vitro. The nucleic acid complex of the disclosure can also be readily introduced into a whole organism to measure the sodium concentration in a cell or tissue in vivo. For example, nucleic acid complex of the disclosure can be transferred into an organism by physical, chemical or biological means, e.g., direct injection.

In certain embodiments, the methods for introducing nucleic acid complexes of the disclosure may be those disclosed in Chakraborty et al., “Nucleic Acid-Based Nanodevices in Biological Imaging,” Annu. Rev. Biochem. 85:349-73 (2016), incorporated in its entirety by reference herein.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. One colloidal system for use as a delivery vehicle in vitro and in vivo is a liposome (i.e., an artificial membrane vesicle). The preparation and use of such systems is well known in the art.

In some embodiments, the use of lipid formulations is contemplated for the introduction of the nucleic acid complex of the disclosure into host cells (in vitro, ex vivo or in vivo). In some embodiments, the nucleic acid complex of the disclosure may be associated with a lipid. The nucleic acid complex of the disclosure associated with a lipid may be encapsulated in the aqueous interior of a liposome, interspersed within the lipid bilayer of a liposome, attached to a liposome via a linking molecule that is associated with both the liposome and the oligonucleotide(s), entrapped in a liposome, complexed with a liposome, dispersed in a solution containing a lipid, mixed with a lipid, combined with a lipid, contained as a suspension in a lipid, contained or complexed with a micelle, or otherwise associated with a lipid. The lipid, lipid/nucleic acid complex compositions are not limited to any particular structure in solution. For example, they may be present in a bilayer structure, as micelles, or with a “collapsed” structure. They may also simply be interspersed in a solution, possibly forming aggregates which are not uniform in either size or shape.

Liposome-mediated oligonucleotide delivery and expression of foreign DNA in vitro has been very successful. Wong et al. (1980) demonstrated the feasibility of liposome-mediated delivery and expression of foreign DNA in cultured chick embryo, HeLa and hepatoma cells. Nicolau et al. (1987) accomplished successful liposome-mediated gene transfer in rats after intravenous injection.

In some embodiments, the one or more nucleic acid complexes of the disclosure are linked to a targeting sequence that directs the nucleic acid complex to a desired cellular compartment.

### Diseases Detection and Monitoring

The methods, compositions, nucleic acid complexes, and kits of the disclosure can be used for the detection of diseases, the monitoring of diseases, and as a drug screening platform. In some embodiments, the disease is characterized as a lysosomal dysfunction disease. In some embodiments, the pathology of the disease includes lysosomal dysfunction.

Lysosomal dysfunction diseases include, for example, autosomal recessive osteopetrosis, Farber disease, Krabbe disease (infantile onset and late onset), Fabry disease (Alpha-galactosidase A), Schindler disease (Alpha-galactosidase B), Sandhoff disease (infantile, juvenile, or adult onset), Tay-Sachs, juvenile hexosaminidase A deficiency, chronic hexosaminidase A deficiency, glucocerebroside, Gaucher disease (Type I, II, and III), lysosomal acid lipase deficiency (early onset and late onset), Niemann-Pick disease (Type A and B), sulfatidosis, metachromatic leukodystrophy (MLD), saposin B deficiency, multiple sulfatase deficiency, mucopolysaccharidoses: MPS I Hurler Syndrome, MPS I S Scheie Syndrome, MPS I H-S Hurler-Scheie Syndrome, Type II (Hunter syndrome), Type III (Sanfilippo syndrome), MPS III A (Type A), MPS III B (Type B), MPS III C (Type C), MPS III D (Type D), Type IV (Morquio), MPS IVA (Type A), MPS IVB (Type B), Type VI (Maroteaux-Lamy syndrome), Type VII Sly Syndrome, Type IX (Hyaluronidase Deficiency); Mucolipidosis: Type I (Sialidosis), Type II (I-cell disease), Type III (Pseudo-Hurler Polydystrophy/Phosphotransferase Deficiency), Type IV (Mucolipidin 1 deficiency); Niemann-Pick disease (Type C and D), Neuronal Ceroid Lipofuscinoses: Type 1 Santavuori-Haltia disease/Infantile NCL (CLN1 PPT1), Type 2 Jansky-Bielschowsky disease/Late infantile NCL (CLN2/LINCL TPP1), Type 3 Batten-Spielmeyer-Vogt disease/Juvenile NCL (CLN3), Type 4 Kufs disease/Adult NCL (CLN4), Type 5 Finnish Variant/Late Infantile (CLN5), Type 6 Late Infantile Variant (CLN6), Type 7 CLN7, Type 8 Northern Epilepsy (CLN8), Type 8 Turkish Late Infantile (CLN8), Type 9 German/Serbian Late Infantile (Unknown), Type 10 Congenital Cathepsin D Deficiency (CTSD); Wolman disease, alpha-mannosidosis, beta-mannosidosis, aspartylglucosaminuria, fucosidosis, lysosomal transport diseases, cystinosis, pycnodysostosis, salla disease/sialic acid storage disease, infantile free sialic acid storage disease (ISSD), glycogen storage diseases, Type II Pompe Disease, Type IIIb Danon disease, and cholesteryl ester storage disease. In some embodiments, the disease is autosomal recessive osteopetrosis. In some embodiments, the disease is Niemann-Pick C disease.

### Kits

The materials and components described for use in the methods may be suited for the preparation of a kit. Thus, the disclosure provides a detection kit useful for determining the pH and the presence, absence, or concentration of Na⁺ in a sample, cell or region thereof. Specifically, the technology encompasses kits for measuring the Na⁺ of one or more cells in a sample. The disclosure also provides a detection kit useful for determining the presence, absence, or concentration of Na⁺ in a sample, cell or region thereof. Specifically, the technology encompasses kits for measuring the Na⁺ of one or more cells in a sample. For example, the kit can comprise a nucleic acid complex as described herein.

In some embodiments, the methods described herein may be performed by utilizing pre-packaged diagnostic kits comprising the necessary reagents to perform any of the methods of the technology. For example, such a kit would include a detection reagent for measuring the Na⁺ of a cell or region thereof, or a detection reagent for measuring the Na⁺ of a cell or region thereof. In one embodiment of such a kit, the detection reagents are the nucleic acid complexes of the disclosure. Oligonucleotides are easily synthesized and are stable in various formulations for long periods of time, particularly when lyophilized or otherwise dried to a powder form. In this form, they are easily reconstituted for use by those of skill in the art. Other reagents and consumables required for using the kit could be easily identified and procured by those of skill in the art who wish to use the kit. The kits can also include buffers useful in the methods of the technology. The kits may contain instructions for the use of the reagents and interpreting the results.

In some embodiments, the technology provides a kit comprising at least one sample (e.g., a Na⁺ concentration standard) packaged in one or more vials for use as a control. Each component of the kit can be enclosed within an individual container and all of the various containers can be within a single package, along with instructions for performing the assay and for interpreting the results of the assays performed using the kit.

In some embodiments, the kit comprises a device for the measurement of Na⁺ in a sample. In some embodiments, the device is for measuring Na⁺ in cell culture or in whole, transparent organisms (e.g., C. elegans).

### EXAMPLES

Certain aspects of the disclosure are illustrated further by the following examples, which are not to be construed as limiting the disclosure in scope or spirit to the specific methods and materials described in them.

### Materials and Methods

Reagents. All oligonucleotides (Table 1) used in this study were purified by high performance liquid chromatography (HPLC) were purchased from Integrated DNA Technologies (USA). All oligonucleotides were subjected to ethanol precipitation and quantified using UV absorbance. ¹H NMR and ¹³C NMR spectra of the newly synthesized compounds were recorded on a Bruker AVANCE II₊, 500 MHz NMR spectrophotometer in CDCl₃ and tetramethylsilane (TMS) was used as an internal standard. Mass spectra were recorded with an Agilent 6224 Accurate-Mass time-of-flight (TOF) liquid chromatography-mass spectrometry (LC/MS). Streptavidin-coated microspheres were purchased from Bangs Laboratories, Inc., Gramicidin, nigericin, and monensin were purchased from Cayman Chemicals. All other reagents were purchased from Sigma-Aldrich (USA) unless otherwise specified.

### Conjugation of CG to DNA

5′-amine modified single strand DNA (IDT) was reacted overnight with 20 equivalence of azide-(PEG)₄-NHS ester (Click Chemistry Tool, AZ103) in 100 mM Na₂HPO₄ buffer with pH adjusted to ˜8.5 by adding NaHCO₃. Ethanol precipitation of DNA was done by adding 0.1 volume of 3 M NaOAc, and 3 volume of molecular biology grade ethanol. Reaction mix was kept at −20° C. for at least 2 h to allow DNA to precipitate then centrifuged at >12000 g to collect DNA precipitate. DNA was redissolved in 50 mM pH 7.0 phosphate buffer after wash with 70% ethanol.

To confirm full conversion of amine to azide, a small aliquot of azide-DNA was reacted with 2 equivalences of 5 kDa mPEG-DBCO (Nanocs, PG1-DB-5K) in 50 mM pH 7.0 phosphate buffer overnight and run on 12% native PAGE. PEGylated DNA will have way less gel shift and no azide-DNA band should be seen. Azide-DNA can be made in bulk and aliquoted in −20° C. for 12 months.

CuAAC reaction was used for CG conjugation to azide-DNA. Copper catalyst was prepared by premixing concentrated CuSO₄ and THPTA (100 mM, 10 eq.). Azide-DNA (1.3 mM, 1 eq.) was purged with nitrogen for 1 min. Propargyl-CG (2.6 mM in DMSO, 2 eq.), CuSO₄/THPTA mix, and sodium ascorbate (1 M, 40 eq.) were fully dissolved in 30% DMSO in buffer. Reaction mix was briefly purged with N₂ and tube was sealed and allow to react for >5 h. DNA was precipitated and tested by both 12% native PAGE and UV-Vis.

Na⁺ sensitivity by fluorescence spectroscopy

100 nM of RatiNa was aliquoted in UB buffer (10 mM HEPES, MES and potassium acetate, 140 mM NaCl/KCl). pH adjusted by HCl or KOH) Fluorescent emission spectra was taken by Fluoromax (Horiba) with the following collection parameters: For CG, dex=522 nm, range from 530 nm to 600 nm. For ATTO647N, dex=645 nm, range from 660 nm to 700 nm.

### RatiNa^{biotin} Conjugation to Streptavidin Coated Bead

1 μm streptavidin coated polystyrene bead (Bangs Laboratories, CP01004) was first washed twice with 50 mM pH 7.4 phosphate buffer by centrifuging at 5,000 rpm. The bead was then incubated with 10 μM of RatiNa^{biotin} in 50 mM potassium phosphate buffer, pH 7.4. Either vigorous shaking or magnetic stirrers can be used to keep beads well mixed. After 2 h of shaking the bead can be collected by centrifuging at 5,000 rpm and stored in 50 mM potassium phosphate buffer, pH 7.4. 0.1% of Tween-20 was added to prevent aggregation of beads.

### Na⁺ Sensitivity by Bead Imaging

RatiNa beads were resuspended in clamping buffer (1 mM to 2 M NaCl, 10 mM HEPES, 10 mM MES, 10 mM potassium acetate) with varying concentration of [Na⁺] (1 mM to 2 M) and pH (4.5-7.5). The bead solution was drop casted on poly-D-lysine coated glass bottom dishes (Cellvis D35-14). Poly-D-lysine help with immobilization of the beads. Bead was imaged by either wide field microscope (Olympus IX83) or confocal microscope (Leica Stellaris 8). Individual bead was analyzed by taking intensity in CG channels and ATTO channels. Average G/R for >100 beads were calculated and normalized to the lowest average G/R in all samples.

### Na⁺ Selectivity by Bead Imaging

For selectivity test, fold change of RatiNa signal was compared to the fold change of 5 mM to 145 mM Na⁺. RatiNa beads were resuspended in 5 mM Na⁺ buffer (5 mM NaCl, 10 mM HEPES, 10 mM MES, 10 mM potassium acetate, pH 7.4) and images were taken in both CG and ATTO channels in wide field microscope. Then one of the extra cations or osmolyte (145 mM KCl/10 mM LiCl/10 mM CaCl₂)/10 mM MgCl₂/300 mM NMDG) is added to the buffer to indicated final concentration. Bead signal was imaged again, and fold change was calculated by dividing G/R after and before adding other cations or osmolytes.

### Colocalization Analysis

Colocalization analysis is used to confirm targeting to specific organelle. In C. elegans, endosome and lysosome markers are membrane bound and vesicles are big in size with >3 μm average diameter. Pixel based colocalization PCC is not suitable and instead number of DNA containing compartment was counted. Percentage of DNA containing compartment having membrane marker is calculated for targeting specificity. In RAW macrophages, lysosomes are smaller and luminal marker is used. Therefore, pixel-based PCC analysis is used for targeting specificity. ImageJ plugin Coloc-2 was used for PCC analysis.

Na⁺ clamping in C. elegans

Young adult worms were injected with 2 μM RatiNa in the pseudocoelom following previously published work and transferred to fresh NGM plate⁵. Worms were then transferred to agar pad (2% agar in M9 buffer). 50 μM levamisole was used to anesthetize worms. 1 h after transferring worms back to NGM plate, worms were punctured several times with injection needle and incubated with Na⁺ clamping buffer (150 mM Nat and K⁺, 150 mM CI, 1 mM CaCl₂), 1 mM MgCl₂, 10 mM HEPES, 5 mM glucose, 50 μM monensin, 50 μM nigericin, 10 μM gramicidin, 100 μM ouabain, pH 5.5) for 1 h. Worms were then imaged in confocal microscope in clamping buffer.

Fluorescent microscopy of Imaging RatiNa

For widefield microscope an Olympus IX83 was used with metal halide lamp (X-cite 120Q), 60× objective (Olympus PlanApo N 60×1.42 NA) and Photometric Evolve delta EMCCD camera. For CG channel a 525/30 BP (Semrock FF01-525/30-25) was used as excitation filter, 532 nm dichroic mirror (Semrock Di02-R532-25×36) and 575/40 BP (Chroma ET575/40m) as emission filter. For ATTO647N channel 640/30 BP (Chroma ET640/30×) was used as excitation filter, tri-band dichroic mirror (Chroma 89016bs) and 705/72 BP (Chroma ET705/72m) as emission filter.

For confocal microscope Leica Stellaris 8 was used. White light laser was set to 85% and 63× objective (Leica HC PL APO CS2 63×1.40NA) and Hybrid detectors (Leica HyD X) were used. Sequential scan is used, and pinhole was set to 95.5 μm. For CG 522 nm laser was used for excitation (30% intensity for RAW cells and 10% intensity for C. elegans) and emission was collected from 530 nm to 580 nm (HyD X2 Gain 100). For ATTO647N, 646 nm laser was used for excitation (2% intensity) and emission was collected from 655 nm to 780 nm (HyD X4 Gain 10).

### Image Analysis

All image processing is performed with ImageJ61. For RatiNa beads, thresholded binary images were created from ATTO channel. Then single beads in focus are automatically picked by “Analyze Particles” plugin in ImageJ with circularity >0.8. Rolling ball method was used for background subtraction. G/R value of each bead is obtained by taking ratio of integrated intensity in CG channel and ATTO channel.

For C. elegans, Endosomes and Lysosomes are Selected by Hand in ATTO Channel.

Rolling ball method was used for background subtraction. G/R value of each endosome or lysosome is calculated by taking ratio of integrated intensity in CG channel and ATTO channel.

For RAW macrophages, lysosomes are selected by a homemade Matlab program which picks out bright punctate ROI in images. Rolling ball method was used for background subtraction. G/R value of each lysosome is calculated by taking ratio of integrated intensity in CG channel and ATTO channel.

Na⁺ heatmap is generated from background subtracted images in CG and ATTO channels. Linear fit equation of G/R to Na⁺ is applied using both images. Result image was pseudo coloured with Vridis lookup table.

### Cell Culture

RAW macrophage (ATCC, TIB-71) was grown according to the manufacturer's protocol. In brief, Cells were grown in DMEM medium (Gibco, 11995) with 10% heat inactivated FBS (Gibco, 26140) and 100 U/mL Pen Strep (Gibco, 15140). Cells were maintained at 37° C. in a humidified chamber at 5% CO₂ concentration. Gentle scraping is used to passage cells. All the experiments were performed on cells at least 24 h after passaging and at confluency ˜50%.

### RAW Cell Stability Assay

Macrophage lysosomes are highly degradative and RatiNa can get digested by DNAse. If DNA backbone is digested and fluorophores can leave the lysosome causing signal loss. TMR-Dextran is stable and will not get digested and there will not be signal loss of TMR. If lysosomes are labeled with both RatiNa^{AT} and TMR-dextran the signal ratio of ATTO to TMR (R/G) is a good indicator of ATTO signal loss and DNA degradation. RAW macrophage lysosomes are labeled with 1 mg/mL TMR dextran with 1 h pulse and 16 h chase. 500 nM RatiNa^{AT} is pulsed for 2 h for maximum uptake. And cells are imaged at different chase time from 30 min to 16 h with wide field microscope. Lysosomal R/G is calculated at each time point. Previously, it has been shown that for 30 min chase there is minimal DNA digestion³⁰. Therefore, DNA integrity can be calculated by comparing average R/G values in each time point normalized to 30 min chase.

Na⁺ clamping in RAW cells

RatiNa sensor was first targeted to lysosomes followed by ionophore treatment to change lysosome lumenal Na⁺ concentration to known extracellular concentrations. Na⁺ clamping buffers (150 mM NaCl/KCl, 1 mM CaCl₂), 1 mM MgCl₂, 10 mM HEPES, 5 mM glucose, pH 5.5) recapitulate cell internal osmotic pressure (321 Osm) to prevent changing of lysosome volume from ion exchanges. 50 μM monensin, 50 μM nigericin, 10 μM gramicidin are used as Na⁺ and K⁺ ionophores to clamp Na⁺ and K⁺ and 100 μM of ouabain is used to inhibit cell surface Na⁺/K⁺ ATPase activity to facilitate clamping⁴. In short, cells were treated with 1 μM RatiNa with 2 h pulsing and 30 min chasing. Then cells were washed with clamping buffer twice and incubated in Na⁺ clamping buffer for 1 h at RT. Cells were then imaged in confocal microscope in clamping buffer to record fluorescence readings from lysosomes.

### Lysosome Targeting in RAW Cells

0.5 mg/mL TMR dextran was pulsed to RAW macrophage for 1 h in Opti-MEM (Gibco, 31985070) and chased for 16 h in complete DMEM to label lysosomes. Then RatiNa^{AT} was pulsed for 2 h in Opti-MEM and chased for 30 min in complete DMEM. Live cells were then imaged in FluoroBrite (Gibco, A1896701) with confocal microscope (Leica Stellaris 8) in both TMR channel and ATTO channel.

### RatiNa Treatment of RAW Macrophage

1 μM RatiNa in prewarmed Opti-MEM was added to RAW macrophage and incubated for 2 h for maximum RatiNa uptake. Cells were then washed 3 times with PBS and incubated in prewarmed complete DMEM medium for 30 min to allow lysosome targeting of RatiNa. RatiNa in cell was then imaged in confocal microscope or widefield microscope.

### Pharmacological Inhibition of Macrophages

RAW macrophage was treated with 100 nM apilimod for 1 h to inhibit PIKfyve. RatiNa treatment was done as stated previously with 100 nM apilimod in all medium. Cell was then imaged in confocal microscope.

C. elegans Strains and Maintaining

Standard methods were followed for the maintenance of C. elegans. Wild type strain used was C. elegans isolated from Bristol (strain N2). Mutant strains were provided by the CGC, which is funded by NIH Office of Research Infrastructure Programs (P40 OD010440).

RRID:WB-STRAIN:NP1129 cdls131 [pcc1:GFP:rab-5+unc-119 (+)+myo-2p::GFP], a transgenic strain that expresses GFP-fused early endosomal marker RAB-5 inside coelomocytes.

RRID:WB-STRAIN:NP871 cdls66 [pcc1:GFP:rab-7+unc-119 (+)+myo-2p::GFP], a transgenic strain that expresses GFP-fused late endosomal/lysosomal marker RAB-7 inside coelomocytes.

RRID:WB-STRAIN:RT258 pwls50 [Imp-1::GFP+Cbr-unc-119 (+)], a transgenic strain expressing GFP-tagged lysosomal marker LMP-1.

RRID:WB-STRAIN:RB836 ok661 is a deletion mutant with 1411 bp deletion in nhx-5 gene (F57C7.2) and does not express NHX-5 globally.

RRID:WB-STRAIN:RB793 ok583 is a deletion mutant with 1702 bp deletion in nhx-7 gene (K09C8.1) and does not express NHX-7 globally.

RRID:WB-STRAIN:RB770 ok549 is a deletion mutant with 1584 bp deletion in nhx-8 gene (Y18D10A.6) and does not express NHX-8 globally.

RRID:VC40914:gk879849 is a substitution mutant R634 to stop codon in ncx-2 gene (C10G8.5).

C. elegans qRT-PCR

Total RNA was isolated from >50 young adult worms. In brief, worms are washed with M9 buffer twice and collected by centrifuging at 1,000×g for 1 min. Aspirate off M9 and add 500 μL Trizol (Thermo 15596026) to the worm. The tube was then flash freezed with liquid N₂ and thawed with 37° C. heat block 5 times followed by 1 min centrifugation at 3,000×g. Top solution was transferred to a new tube and 100 μL chloroform was added. Vortex and wait for aqueous layer to separate from organic layer. Collet aqueous layer and add 250 μL of cold isopropanol. Incubate at room temperature for 5 min and −20° C. for 1 h. Centrifuge at 12,000×g for 10 min at 4° C. and discard supernatant. RNA pellet was washed with 500 μL cold 70% EtOH and centrifuged at 12,000×g for 10 min. Remove all EtOH by carefully pipetting out the supernatant and air-dry pellet with clean N2 or air stream. Resuspend RNA in 30 μL RNAse free water.

cDNA was synthesized with Maxima H Minus cDNA synthesis master mix (Thermo, M1661) according to manufacturer's protocol. qPCR was performed with Roche LightCycler 96. ΔΔC_{T} was used to calculate fold change difference of RNA level compared to control gene ACT-1. Following primers were used for qRT PCR:

NHX-5 fwd:
[SEQ ID NO: 6]
CGT CAA CTG TAG CAG GTT CTA A
NHX-5 rev:
[SEQ ID NO: 7]
GGA AAC GTA GGT GAG GAG TAT G
NHX-7 fwd:
[SEQ ID NO: 8]
GGA GCT TTA CCA CAC GAC TTA T
NHX-7 rev:
[SEQ ID NO: 9]
GTG CAT GAG CTG ACG AAT AGA
NHX-8 fwd:
[SEQ ID NO: 10]
CCA TCG TTC AAC TCG TTA CCT
NHX-8 rev:
[SEQ ID NO: 11]
GAG CAA TGC ACT CAA CAA TCC
NCX-2 fwd:
[SEQ ID NO: 12]
GAT TGA TCG GAG GAG GAG ATA TTG
NCX-2 rev:
[SEQ ID NO: 13]
GTA GTG AGC TGG ATC CAA GAA G
ACT-1 fwd:
[SEQ ID NO: 14]
CGA GCG TGG TTA CTC TTT CA
ACT-1 rev:
[SEQ ID NO: 15]
CTT CTG CAT ACG ATC AGC AAT T

C. elegans Targeting and Colocalization

C. elegans microinjection was performed following previously published³³. For targeting colocalization assay, Worms with fluorescent marker for early endosome, late endosome and lysosome are microinjected with 1 uM of RatiNa^{AT}. Worms were transferred to fresh NGM plate after injection and imaged with confocal microscope after 5 min, 17 min and 60 min for early endosome, late endosome and lysosome targeting respectively. Anti-colocalization was performed similarly but with different time points. Targeting efficiency was evaluated by counting total number of DNA containing endosomes and the ratio of DNA containing endosomes having the fluorescent marker.

C. elegans Adaptation Assay

C. elegans adaptation assay was performed following previously published work⁴¹. In brief, High Na⁺ containing plate was made by supplementing NaCl to NGM plate (51 mM NaCl for normal NGM plate, 200 mM, 300 mM and 400 mM NaCl for high Na⁺ plate). High Na⁺ plates are sealed with parafilm and kept in 4° C. fridge until use. For adaptation, gravid worms were transferred to 200 mM Na⁺ plate. Hatched worms were allowed to grow on 200 mM Na⁺ plate for adaptation. Worms grown to L4 embryonic stage are considered adapted to Na⁺.

For brood size assay, 5 untreated worms and adapted worms are transferred to 200 mM, 300 mM and 400 mM Na⁺ plate at L4 stage. Worms were allowed to lay egg for 24 h and removed from the plate. Progeny was allowed to grow for another 48 h for easier visualization. Plates were photographed and brood size was counted.

### Data Analysis

Numerical data was processed and plotted with Origin. For Na⁺ measurement, single endosomal Na⁺ was represented as a hollow circle data point colour coded with different trials. A filled circle represents mean value of all endosomal Na⁺ level in the indicated trial. A mean line was shown to represent overall average endosomal Na⁺. And error bar reports 1 S.D. For statistical analysis, paired t-test is used.

### Preparation of Na⁺ Fluorophore

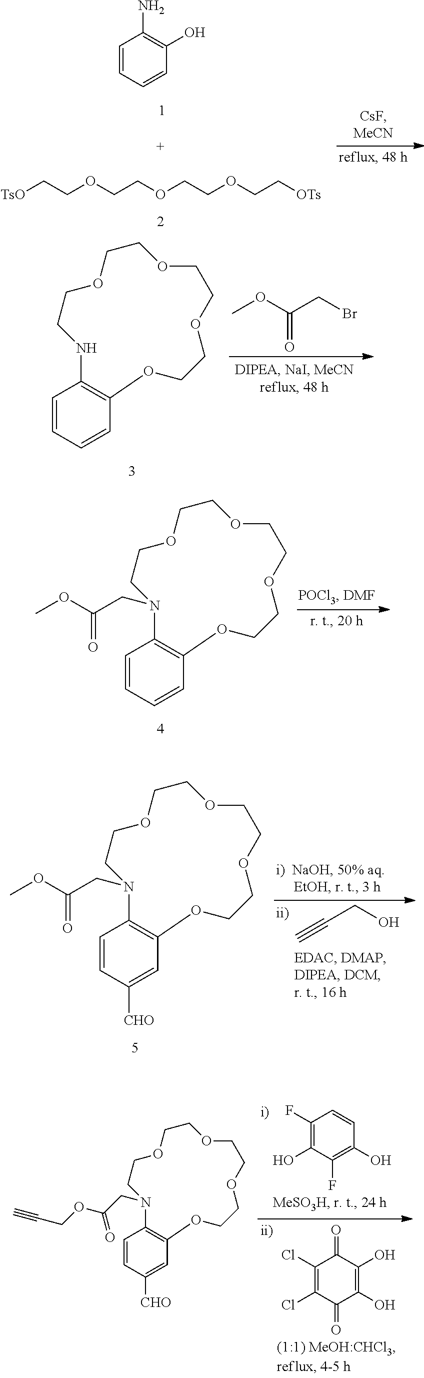

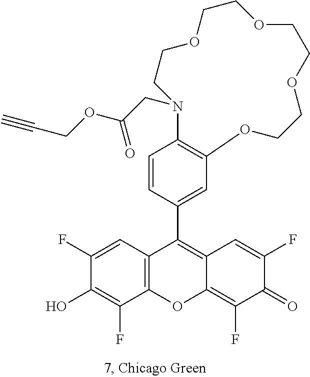 [SMILES: C#CCOC(=O)CN1CCOCCOCCOCCOc2cc(-c3c4cc(F)c(=O)c(F)c-4oc4c(F)c(O)c(F)cc34)ccc21]

Compounds 3-5 were synthesized following previously reported methods²⁴:

Synthesis of compound 3. A mixture of 2-aminophenol, 1 (1 g, 9 mmol) and cesium fluoride (7 g, 45 mmol) in acetonitrile (800 mL) was stirred vigorously for 2 h under nitrogen atmosphere. Next, tetraethylene gycol di (p-toluenesulfonate), 2 (4.57 g, 3.6 mL, 9 mmol) in 50 mL acetonitrile was added to the solution and refluxed under nitrogen atmosphere for 48 h. The solution was allowed to cool down to room temperature and then evaporated using a rotavac. The residue was dissolved in chloroform (600 mL) and filtered. The chloroform layer was then washed with water (200 mL), saturated sodium bicarbonate (200 mL), brine (200 mL), and evaporated using a rotavac. The residue was subjected to silica gel column chromatography (50-70% ethyl acetate/hexane) to obtain pure 3 (1.1 gm, 4.1 mmol, yield ˜45%) as a yellowish-brown oil. ESI-MS: calculated m/z for [3+H⁺]=268.15, observed m/z=268.2. ¹H NMR (400 MHZ, CDCl₃, 0 ppm): 6.89-6.85 (t, 1H, J₁=J₂=8 Hz), 6.77-6.75 (d, 1H, J=8 Hz), 6.64-6.58 (dd, 2H, J₁=16 Hz, J₂=J₃=8 Hz), 5.13 (b, 1H), 4.12-4.09 (m, 2H), 3.86-3.84 (t, 2H, J₁=J₂=4 Hz), 3.80-3.77 (t, 2H, J₁=4 Hz, J₂=8 Hz), 3.73-3.67 (m, 8H), 3.26-3.24 (t, 2H, J₁=J₂=4 Hz). ¹³C NMR (100 MHz, CDCl₃, 0 ppm): 146.4, 139.6, 121.9, 116.4, 112.0, 110.3, 70.4, 70.0, 69.9, 69.5, 69.0, 68.3, 43.3. ESI-MS (m/z for [3+H⁺]): Calculated, 268.15; Observed, 268.2.

Synthesis of compound 4. Compound 3 (1.0 μm, 3.7 mmol), methyl bromoacetate (1.14 g, 0.7 mL, 7.5 mmol), DIPEA (2.4 g, 3.3 mL, 18.7 mmol) and sodium iodide (0.55 g, 3.7 mmol) were taken in dry acetonitrile (30 mL) and refluxed for 48 h under nitrogen atmosphere. The reaction mixture was allowed to reach room temperature and the solvent was evaporated using a rotavac. The residue was dissolved in dichloromethane (100 mL) and washed with water (20 mL). The organic layer was dried with anhydrous magnesium sulfate, filtered and evaporated using a rotavac. The crude product was subjected to silica gel column chromatography (0-8% methanol/dichloromethane) to obtain compound 4 (0.9 g, 2.6 mmol, yield ˜71%). ¹H NMR (400 MHZ, CDCl₃, 0 ppm): 6.97-6.94 (m, 1H), 6.89-6.85 (m, 2H), 6.82-6.80 (m, 1H), 4.14-4.12 (m, 4H), 3.90-3.88 (m, 2H), 3.77-3.73 (m, 4H), 3.70-3.66 (m, 11H).

Synthesis of compound 5. POCl₃ (1.97 g, 1.2 mL, 12.9 mmol) was slowly added to dry DMF (1 mL) under ice-cold condition and nitrogen atmosphere to form the Vilsmeier's reagent. Compound 4 (0.9 g, 2.64 mmol) dissolved in dry DMF (0.7 mL) was slowly added to the Vilsmeier's reagent and the reaction mixture was stirred for 24 h at room temperature under nitrogen atmosphere. The solution was then slowly poured into a mixture of ice-cold saturated potassium carbonate solution (50 mL). Water (25 mL) was added and the aqueous solution was extracted with dichloromethane (20 mL, 5×). The combined organic extract was dried with anhydrous sodium sulfate, filtered and evaporated using a rotavac. The crude product was subjected to silica gel column chromatography (0-8% methanol/dichloromethane) to obtain compound 5 (0.74 g, 2.0 mmol, yield ˜76%) as a yellow oil. ¹H NMR (400 MHZ, CDCl₃, 0 ppm): 9.75 (s, 1H), 7.35-7.32 (m, 2H), 6.79-6.77 (d, 1H, J=8 Hz), 4.26 (s, 2H), 4.21-4.19 (m, 2H), 3.91-3.87 (m, 4H), 3.72 (s, 3H), 3.69-3.65 (m, 10H). 13C NMR (100 MHz, CDCl₃, 0 ppm): 190.0, 171.0, 149.8, 145.8, 128.8, 126.0, 116.4, 111.0, 70.6, 69.9, 69.7, 69.3, 68.9, 68.4, 53.1, 52.0, 51.4. ESI-MS (m/z for [5+H⁺]): Calculated, 368.17; Observed, 368.1.

Synthesis of compound 6. Compound 5 (0.74 g, 2.0 mmol) and sodium hydroxide (0.24 g, 6.0 mmol) were dissolved in 50% aqueous ethanol (8 mL) and stirred at room temperature for 2.5 h. Next, the solution was neutralized with 3N HCl and ethanol was evaporated using rotavac. The concentrate was diluted with water (30 mL) and extracted with dichloromethane (20 mL, 5×). The combined organic extracts were washed with brine (20 mL, 1×), dried over anhydrous sodium sulfate, filtered, and evaporated to dryness using a rotavac. The obtained residue (0.55 g) was used in the next step without any purification. The crude residue, along with propargyl alcohol (0.13 g, 0.13 mL, 2.31 mmol), DIPEA (0.3 g, 0.4 mL, 2.3 mmol), and DMAP (37 mg, 0.31 mmol) were dissolved in dry dichloromethane (10 mL) and the solution was cooled in an ice bath. EDC (0.44 g, 2.3 mmol) was added to it and the reaction was stirred at room temperature for 16 h. On reaction completion, the solution was diluted with dichloromethane (30 mL), washed with water (20 mL, 2×) and brine (20 mL, 2×), dried over anhydrous sodium sulfate, filtered, and evaporated using a rotavac. The obtained crude residue was subjected to silica gel column chromatography (0-5% methanol/dichloromethane) to obtain 6 (0.46 g, 1.2 mmol, ˜59% yield) as a yellow viscous oil. ¹H NMR (400 MHZ, CDCl₃, 0 ppm): 9.77 (s, 1H), 7.36-7.33 (m, 2H), 6.83-6.81 (d, 1H, J=8 Hz), 4.74-4.73 (d, 2H, J=4 Hz), 4.35 (s, 2H), 4.22-4.20 (m, 2H), 3.91-3.88 (m, 4H), 3.72-3.66 (m, 11H). ¹³C NMR (100 MHZ, CDCl₃, δ ppm): 189.6, 169.3, 149.3, 144.9, 128.4, 125.7, 116.3, 110.3, 76.3, 74.4, 70.0, 69.6, 69.4, 69.2, 69.1, 68.6, 68.2, 67.6, 52.5, 51.5, 51.2. ESI-MS (m/z for [6+H⁺]): Calculated, 392.17; Observed, 392.2.

Synthesis of compound 7. Compound 6 (132 mg, 0.34 mmol), 2,4-difluororesorcinol (98.5 mg, 0.67 mmol) and methanesulfonic acid (3 mL) was stirred at room temperature for 48 h under nitrogen atmosphere. The reaction mixture was diluted with water (2 mL) and slowly poured into ice-cold sodium acetate solution (3N, 15 mL) and extracted with ethyl acetate (20 mL, 5×). The combined organic extract was dried with anhydrous sodium sulfate, filtered and evaporated using a rotavac. The obtained crude solid (200 mg) and chloranil (300 mg, 1.2 mmol) was taken in 1:1 chloroform/methanol (15 mL) and refluxed for 4-5 h. The reaction mixture was cooled, filtered and evaporated using a rotavac. The residue was subjected to reverse phase HPLC using 1:1 methanol/acetonitrile as the eluent to obtain 7 (2.5 mg, 3.9 nmol, yield ˜11%). QTOF-HRMS (m/z for [7+H⁺]): Calculated, 646.1695; Observed, 646.1688.

### CG Conjugation and Validation

25mer 5′-amine with C6 linker modified DNA was purchased (IDT) and quantified by 260 nm absorption. ssDNA-NH₂ was converted to ssDNA-N₃ by NHS ester activated crosslinking. DNA was then ethanol precipitated from reaction mix and washed with 70% EtOH. In 12% native PAGE ssDNA-NH₂ and ssDNA-N₃ have similar gel shift as seen in FIG. 11B. CG dye CuAAC coupling to ssDNA-N₃ was done by first premixing 10 mM/20 mM CuSO₄/THPTA for ligand chelation. Add copper catalyst mix to DNA and propargyl CG. Finally add freshly dissolved 100 mM sodium ascorbate and purge the reaction mix with nitrogen gas for 3 min. Wrap the tube with parafilm and stir to react overnight at room temperature. Reaction mix was taken at 5 hr and 20 hr and ran by 12% native PAGE. Fluorophore coupling was confirmed by the lower fluorescent DNA band. Upper band in fluorescence channel disappeared after ethanol precipitation. The precipitate has intense pink color.

To confirm full conversion of ssDNA-NH₂ to ssDNA-N₃, a small aliquot of ssDNA-N₃ was taken and reacted with 10 k Da DBCO-PEG to make ssDNA-PEG. This strategy was used because DBCO and azide can react to completion and bulky 10 k Da PEG can alter the gel shift (FIG. 11C).

To confirm full conversion of ssDNA-N₃ to ssDNA-CG, absorption spectra was taken for the conjugate (FIG. 12A). €=251,000 L/(mole·cm) for 25 mer ssDNA at 260 nm. €=21,000 L/(mole·cm) for CG at 522 nm. All free CG dye is excluded from EtOH precipitation and the calculated molar ratio of CG peak and DNA peak shows is ˜1:1.

### PAGE Analysis of RatiNa Sensors

12% native PAGE was used to check the annealed RatiNa sensors. Gel was run at 100 V for 45 minutes. All three single strands and partial annealed products are run together. All three single strands have significantly different gel shift and not present in the annealed sample. The partial annealed products have a slight gel shift compared to RatiNa and both products are not seen in annealed sample. And the annealed sensor contains both CG dye and normalizing dye by showing both red and green fluorescence.

Biotinylated RatiNa sensor was made with same two fluorescent ssDNA and a 3′-biotin modified ssDNA (IDT). Similarly, RatiNa^{biotin} contains both CG dye and normalizing dye by showing both red and green fluorescence and no partially annealed products.

### Na⁺ and pH Response of RatiNa on Beads

To fully characterize RatiNa in terms of Na⁺ affinity, pH insensitivity and selectivity, it was decided to use fluorescent imaging method since the fluorescence signal is the final read out. Thus, RatiNa can be imaged with same image acquisition settings as used for both in vitro characterization and in vivo measurement. To image RatiNa, the biotinylated RatiNa was used to coat streptavidin bead with RatiNa and image the bead in various buffer conditions. In brief, 1 μm streptavidin coated polystyrene bead (Bangs Laboratories, CP01004) was first washed twice and then incubated with 10 μM of RatiNa^{biotin} in phosphate buffer. Either vigorous shaking or magnetic stirrers can be used to keep beads well mixed. After 2 hours of shaking the bead can be collected by centrifuging at 5,000 rpm and stored in phosphate buffer. 0.1% Tween-20 was added to prevent aggregation of beads.

Beads were then imaged to test Na⁺ response. RatiNa beads were resuspended in Na⁺ buffer (1 mM to 2000 mM NaCl, 10 mM HEPES, 10 mM MES, 10 mM KOAc. pH from 4.5 to 7.4 adjusted by HCl or KOH) and added to poly-D-lysine coated glass bottom dishes (Cellvis D35-14). Poly-D-lysine coating help with immobilization of the beads. Bead was imaged by wide field microscope. Individual bead was first picked with Analyze Particle function in FIJI and saved as individual ROI. Then each ROI was analyzed by taking integrated intensity in CG channels and ATTO channels. Average G/R for >100 beads were calculated and normalized to the lowest average G/R in all samples. It was noticed that on the log scale normalized G/R can be approximated with a sigmoidal curve (FIG. 5D). This is expected response for binding-based fluorescent sensor.

For pH insensitivity, the sigmoidal curve at each pH from 4.5 to 7.4 was compared. From the overlay and K_{d} calculations, it was concluded that both fold change and K_{d} are constant from acidic to neutral pH (FIG. 5E). Since the lysosome is most acidic organelle with average pH of 4.52. RatiNa can reliably measure Na⁺ in acidic organelles, and it was expected to see the same Na⁺ response regardless of pH.

C. elegans Na⁺ Clamping

Previous protocol for C. elegans microinjection and clamping was followed²⁹. The C. elegans clamping buffer has pH 5.5 and contains 150 mM Na⁺ and K⁺, 150 mM CI, 50 μM monensin, 50 μM nigericin, 10 μM gramicidin, 100 μM ouabain. The buffer mimicks Na⁺ and K⁺ level in biological system: total amount of both cation is about 150 mM. Monensin is a Na⁺ ionophore, nigericin is a K⁺ ionophore, gramicidin can form ion-channel like pores in membranes to facilitate Na⁺ and K⁺ ion exchange via diffusion⁴. Ouabain inhibits Na⁺/K⁺ ATPase which actively transport Na⁺ and K⁺ to maintain high cytosolic K⁺ and low cytosolic Na⁺. A pH of 5.5 was chosen because it's close to C. elegans lysosome pH. Ionophores binds to Na⁺ or K⁺ in deprotonated form and release bound ion when protonated. Having a steep pH gradient across lysosomal membrane will make the protonation and deprotonation cycle much slower therefore pH 5.5 buffer is used.

Clamping of Na⁺ proved to be challenging since the amount of Na⁺ needs to be transported is in millimolar scale. After one hour of clamping morphological change of C. elegans lysosomes was observed from spherical to amorphous, and the effect is more severe in clamping at extreme values of 5 mM and 145 mM Na⁺. G/R values were compared of clamped lysosomes and RatiNa beads and found indeed G/R level are very similar in both cases with slight shift in extreme values (FIG. 2D). Therefore, the bead data was used for extrapolation of RatiNa calibration curve in C. elegans because at extreme Na⁺ values clamping may not work to the fullest.

### RAW Macrophage Na⁺ Clamping and Measurement

Unlike C. elegans coelomocyte which can accumulate almost all injected DNA in lysosomes, cultured cells uptake less DNA and the overall fluorescence signal is weaker. A new calibration curve is required for measurements in macrophage lysosomes. The clamping buffer for macrophage used is pH 5.5, 150 mM Na⁺ and K⁺, 150 mM CI, 5 mM D-glucose, 1 mM MgCl₂, 1 mM CaCl₂), 10 mM HEPES, 50 μm monensin, 50 μm nigericin, 10 μm gramicidin, 100 μm ouabain⁶. The buffer is slightly different from C. elegans clamping buffer and better recapitulate internal osmolarity of cultured cells, prevent cells from swelling or shrinking. With similar workflow macrophage lysosomes were clamped and attempted to image RatiNa in Na⁺ clamped lysosomes. It was noticed that CG signal is very low and G/R fold change from lysosomes are only ˜2 fold compared to ˜4 fold from C. elegans measurements.

In order to show that RatiNa can reliably measure Na⁺ in macrophage lysosomes, the difference of RatiNa fold change in macrophages was investigated. First, it was observed that autofluorescence can be detected at the optimal excitation and emission wavelength of CG in macrophages (FIG. 6B) Then, RatiNa^{AT} in macrophages were observed in both CG channel and ATTO channel. The observed signal in both channels and if ROI is picked from ATTO channel and G/R is calculated for RatiNa^{AT} containing lysosomes, a non-specific G/R signal is obtained even without the CG dye (FIG. 6D). And if the averaged non-specific G/R signal is subtracted from G/R from clamped lysosomes, the fold change of ˜3.5 fold from 5 mM to 145 mM Na⁺ can be extracted, which is similar to C. elegans clamped lysosomes. It was concluded that even though fold change is less in macrophages, a linear calibration curve can still be obtained with the Na⁺ clamping value. The new calibration line should be used for all RAW macrophage lysosome Na⁺ measurement.

### RatiNa Stability Assay in Macrophage Lysosomes

Lysosome is highly degrading and harbors lots of endonuclease and exonucleases⁶³. Therefore, there was a need to test integrity of DNA backbone of RatiNa and find out the time when DNA is significantly degraded. Lysosomes were first labeled with TMR-dextran which is not digested by lysosome enzymes. Then RatiNa^{AT} was targeted to lysosome with 2 h pulse and 30 min chase. Green signal from TMR-dextran will stay constant and red signal from RatiNa^{AT} will decrease as DNA degrades and free dye leaves lysosome via diffusion. Therefore R/G is a good indication of DNA degradation. Indeed, a decrease of overall red signal was observed (FIG. 6F). The single lysosomal R/G signal was taken at different chase time point and plotted in histogram. (FIG. 6D). The large spread of the R/G value is due to differential uptake of dextran and DNA through fluid phase endocytosis and receptor mediated endocytosis respectively. But the R/G average indeed decreases. Previously it was shown that for 30 min chase there is minimal DNA digestion². To better interpret the data, the following equation was utilized to calculate percentage of undigested DNA (FIG. 6G)

$$lysosomal D N A = \frac{{( \frac{R}{G} )}_{t}}{{( \frac{R}{G} )}_{{t}_{0}}}$$

Where t=chase time and to is 30 min. It was decided to not measure lysosome for more than 30 min chase time to guarantee RatiNa sensor is not degraded.

Colocalization Analysis of RatiNa^{AT} in C. elegans Early Endosome, Late Endosome, and Lysosome

RatiNa targeting organelles through scavenger receptor mediated endocytosis.

Endocytosis is time dependent process and cargo is trafficked to early endosome then to late endosome and finally to lysosome. If RatiNa is imaged at specific chase time, it can be found localized majorly to early endosome, late endosome or lysosomes. It has been previously established that these timepoints in C. elegans with other DNA based sensors^{22,23}. Now, it was needed to determine the effectiveness and specificity of RatiNa targeting using these timepoints. For early endosome targeting cdls131 worm were used which express GFP::RAB-5 fusion protein in coelomocytes. RAB-5 is an early endosome marker protein. 5 min after injecting cdls131 worm with RatiNa^{AT}, worms were imaged and colocalization was determined as described in methods. For late endosome targeting cdls66 mutant worms were used which express GFP::RAB-7 fusion protein in coelomocytes. RAB-7 is a late endosome marker protein. 17 min after injecting cdls66 worms colocalization of RatiNa^{AT} to late endosome is determined. For lysosome targeting pwls50 mutant worms were used which express GFP with LMP-1 promoter. LMP-1 is a lysosome marker protein. 60 min after pwls50 worms colocalization of RatiNa^{AT} to lysosome is determined. As shown in FIG. 7A, RatiNa is localized to early endosome, late endosome and lysosome with high efficacy at 5 min, 17 min and 60 min respectively. Next, anti-colocalization was checked with other endocytic organelles. RatiNa is trafficked from early endosome to late endosome, so it is required to show at 5 min there's no colocalization to late endosomes. Indeed, there is little colocalization of RatiNa with RAB-7 at 5 min post injection (FIG. 7B). Similarly anti-colocalization with early endosome should be shown for late endosome targeting and there is little colocalization of RatiNa with RAB-5 at 17 min post injection. RAB-7 partially labels lysosome, so it is not a great marker for anti-colocalization. Therefore, LMP-1 anti-colocalization was used at 17 min to show specificity of lysosome targeting (FIG. 7B).

C. elegans Na⁺ Adaptation Assay

The goal for a Na⁺ adaptation assay is to identify the significance of lysosomal Na⁺ through phenotypical manifestation. Even with highly perturbed lysosomal Na⁺ in mutant worms, worms can grow with normal life cycle. And there is no obvious defect from lysosome morphology. It is hypothesized that lysosomal Na⁺ defect may become prominent in a condition with Na⁺ stress or overload. It has been found that C. elegans can adapt to high environmental salt through physiological remodeling⁴¹, which allow them to live in lethal level of salt.

First, it was sought to reproduce the adaptation assay with different Na⁺ transporter mutant worms. The workflow of adaptation is described in methods section. It was found that in 400 mM Na⁺ which previously described as lethal worms is still alive albeit with no movement and only reflex when poked with platinum wire. Therefore, it was decided to score the adaptation with a brood size assay instead of survival assay (FIG. 15) 5 L4 worms were transferred to high Na⁺ plates with 200-400 mM Na⁺ and allowed to make progenies. After 24 hours the original worms were removed from the plate and the progenies were allowed to grow into L3-L4 stage for counting. First, it was confirmed that all worms can remodel and adapt to high Na⁺ to some extent. This is shown by the increase of progeny count from untreated to adapted worms. (FIG. 8A) But Na⁺ transporter mutant worms cannot adapt to Na⁺ as good as N2 worms, shown in 400 mM Na⁺ experiment which only N2 worms can adapt to and make progenies. Notably Δnhx-5 worms were the worst at Na⁺ adaptation and had most perturbed lysosomal Na⁺, indicating lysosomal Na⁺ plays an important role in the Na⁺ adaptation process.

### Lysosomal Na⁺ Measurement for NHX Mutant Worms

Several targets have been identified for organelle Na⁺ transporters. NHX protein is Na⁺/H⁺ exchanger and have been previously shown to localize to intracellular compartment, albeit not specific organelles9. NHX deletion mutant worms were obtained from existing library and perform lysosome Na⁺ measurement in mutant worms (FIGS. 9C-9D). It was found that NHX mutant worms have lower lysosomal Na⁺ compared to WT worms.

Δnhx-5 worms show lowest lysosomal Na⁺ level indicating NHX-5 may directly or indirectly facilitate lysosomal Na⁺ import. Best homolog of worm NHX-5 is human NHE9 (40% identity, 61% similarity, aligned with 90% sequence coverage), which is found to be mainly localized to late endosomes with Na⁺ importing functions⁴⁴. And it was hypothesized that NHX-5 is directly transporting Na⁺ into lysosomes. Δnhx-7 worms have decreased lysosomal Na⁺ but not as low as Δnhx-5 worms. Best homolog of worm NHX-7 is human NHE1 (35% identity, 55% similarity, aligned with 77% sequence coverage), which is a plasma membrane Na⁺/H⁺ exchanger. Δnhx-8 worms lysosomal Na⁺ is unaffected from deletion. Best homolog of worm NHX-8 is NHE8 (48% identity, 69% similarity, aligned with 61% sequence coverage), which is found mainly in TGN. It was hypothesized that NHX-8 may transport Na⁺ in Golgi but cannot communicate to lysosomal Na⁺ transport, whereas NHX-7 is transporting Na⁺ across plasma membrane and loss of NHX-7 mediated plasma membrane Na⁺ transport can affect lysosomal Na⁺ through the cytosolic Na⁺ level change.

Δnhx-5 worms showed the most significant changes in lysosomal Na⁺. The early endosome and late endosome Na⁺ were measured in Δnhx-5 worms and found that only lysosomal Na⁺ level is perturbed in mutant, which indicates that although NHE9 the human homolog is a late endosome Na⁺ importer, NHX-5 is likely a lysosomal Na⁺ importer in C. elegans.

### Expression Level of Exchanger Proteins Remains Similar in Adapted Worms

One of the potential reasons of observing the change in lysosomal Na⁺ in adapted mutant worms is due to differential expression of genes which lead to a change in transporting activity and steady state of lysosomal Na⁺. Theory was tested by comparing expression level of nhx genes in N2 worms through qRT-PCR. The adaptation process was found to not affect the mRNA level of investigated genes (FIG. 8C), further supporting that lysosomal Na⁺ change is due to actual directional change of the Na⁺ exchangers.

### Lysosomal Na⁺ Measurement in Adapted Worms

The lysosomal Na⁺ was observed in adapted WT worms and mutant worms. Intriguingly, it was observed all mutant worms have higher lysosomal Na⁺ adapted to high Na⁺ while N2 worms have lower lysosomal Na⁺ adapted to high Na⁺. First, lysosomal Na⁺ level changes as worms undergo adaptation, indicating lysosomal Na⁺ is playing a role in the Na⁺ adaptation process. It is hypothesized that observed direction of lysosomal Na⁺ change in mutant worms is likely due to a directional change of exchangers. During high Na⁺ stress organelle may require to uptake more Na⁺ and the increase of Na⁺ uptake is counteracted by change of direction of Na⁺ exchangers from Na⁺ import to Na⁺ export, highly active Na⁺ export activity from exchangers causes the equilibrium level of organelle Na⁺ to decrease as seen in FIG. 4D. However, without NHX genes C. elegans cannot export the excess Na⁺ and net increase of Na⁺ is observed as seen in FIG. 4E and FIG. 8B. This effect can be so prominent that lysosomal Na⁺ is perturbed in all NHX and even plasma membrane Na⁺/Ca²⁺ exchanger NCX-2 mutants.

### Design and Sequence of RatiNa Sensors

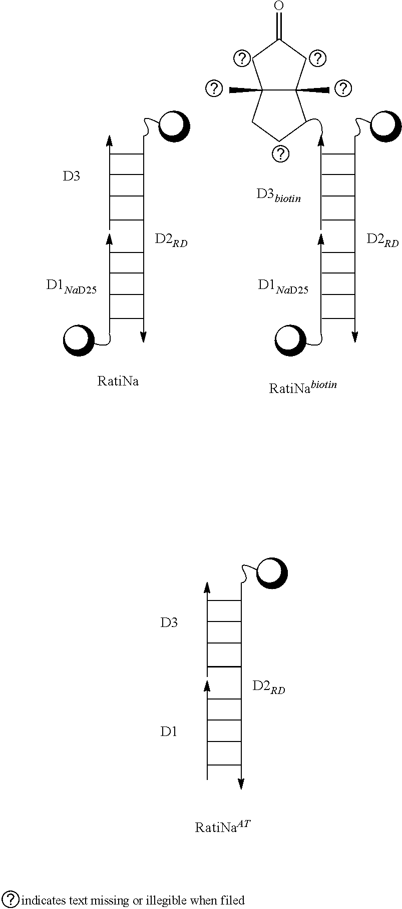

TABLE 1
Design and Sequence of RatiNa sensors

| Name | Sequence |
|---|---|
| D1_{NaD25} | 5′-CG-ATC AAC ACT GCA TAT ATA TAC GAC |
| | C-3′ (25mer) [SEQ ID NO: 1] |
| D1 | 5′-ATC AAC ACT GCA TAT ATA TAC GAC C-3′ |
| | (25mer) [SEQ ID NO: 2] |
| D2_{RDL} | 5′-ATTO647N-C ACT GCA CAC CAG ACA GCA A G |
| | GTC GTA TAT ATA TGC AGT GTT GAT-3′ (45mer) |
| | [SEQ ID NO: 3] |
| D3_{biotin} | 5′-T TGC TGT CTG GTG TGC AGT G-BioTEG-3′ |
| | (20mer) [SEQ ID NO: 4] |
| D3 | 5′-T TGC TGT CTG GTG TGC AGT G-3′ (20mer) |
| | [SEQ ID NO: 5] |

### DISCUSSION

As described herein, a pH-insensitive Na⁺ reporter, denoted RatiNa, that can ratiometrically image intracellular Nat in single organelles in intact cells was developed. RatiNa is a 45-base pair DNA duplex comprising three single-stranded DNA (ssDNA) molecules: a 25-mer strand carrying a Na⁺ sensitive fluorophore for sensing (D1); a 45-mer strand bearing an ion-insensitive internal reference dye for ratiometry (D2), and a 20-mer strand harboring the targeting module that localizes RatiNa in the lumen of specific organelles (D3) (FIG. 1A).

Based on the sensing mechanism of the Na⁺ probe, CoroNa Green24, a novel pH-insensitive fluorophore for D1, denoted Chicago Green (CG, λₑₓ=510 nm, λₑₘ=530 nm) was synthesized (FIGS. 5A and 10A-10B). Like CoroNa Green, CG binds Na⁺ through a 1-aza-15-crown-5 ether moiety and uses photoinduced electron transfer (PET) to switch on or off fluorescence. However, CoroNa Green, is pH sensitive because the aza-crown and hydroxyl groups are protonated under acidic conditions while CG is not. CG is therefore suitable for organelles. Because fluoro substitutions generally lower the pKₐ of fluoresceins25, CG has a tetrafluoro fluorescein core which reduces its pH sensitivity. The Na⁺ binding affinity (K_{d}) of CG was shown to be completely pH independent between pH 4.5-7 (FIGS. 5B-5C). CG is further modified with a propargyl group for conjugation to a 5′-azido substituted D1 using copper-catalyzed azido-alkyne cycloaddition (FIGS. 11A-11D and 12A-12B)26,27. ATTO647N (λₑₓ=646 nm, λₑₘ=663 nm) was chosen as the reference dye on D2 because it is bright, photostable, and insensitive to pH, Na⁺ and other ions28. Because its emission spectrum does not overlap significantly with CG, the ratio of CG (G) to ATTO647N (R) fluorescence (G/R) in RatiNa corrects for CG intensity differences arising from inhomogeneous probe distribution and/or uptake. This correction gives a readout for Na⁺ concentration ([Na⁺]) alone. When D3 hybridizes to D1 and D2, it forms a 45-bp duplex DNA that targets RatiNa to endocytic organelles via receptor mediated endocytosis (FIGS. 13A-13B) 29.30.31.

When attached to RatiNa, CG excitation and emission maxima were red shifted by ˜ 12 nm but its intensity still increased with increasing [Na⁺], while that of ATTO647N was constant (FIGS. 1B-1C). RatiNa was calibrated immobilized on streptavidin-coated beads by fluorescence imaging in buffers of varying pH and [Na⁺] (FIGS. 14A-14B). The three-dimensional surface plot of G/R values versus pH and [Na⁺] shows that the Na⁺ response of RatiNa is pH independent (FIGS. 1D and 5D-5F). Further, RatiNa response is specific to Na⁺ and barely affected by other physiological ions like K⁺, Li⁺, Ca²⁺ and N-methyl-D-glucamine (NMDG) (FIGS. 1E and 5G). Together, these data show RatiNa is a pH-insensitive Na⁺ reporter that is sufficiently specific for measuring Na⁺ in acidic organelles.

Given lysosomes are the only organelles with at least population averaged Na⁺ levels that can be used for validation, RatiNa was used to map lysosomal Na⁺ in cultured murine macrophages and in vivo in coelomocytes of C. elegans. Both these systems are amenable to analysis with DNA nanodevices because they express scavenger receptors abundantly, whose cognate ligand is duplex DNA30.32.33. To track RatiNa trafficking and determine when it reaches lysosomes in both systems, a RatiNa lacking CG was used and carrying only the ATTO647N dye (RatiNa^{AT}). LMP1-GFP worms were used whose lysosomes are labeled with green fluorescence protein (GFP), and RAW 264.7 macrophages whose lysosomes are labeled with TMR dextran. In LMP1-GFP worms, RatiNa^{AT} colocalized with GFP-labeled lysosomes 1 h post-injection (FIG. 2A). Pulsing RAW 264.7 cells with 500 nM RatiNa^{AT} for 2 h followed by a 30 min chase led to robust colocalization with the TMR-labeled lysosomes. Because lysosomes of murine macrophages are more degradative than those in nematodes33, the stability of RatiNa^{AT} in macrophages was used and found that the fluorescence of RatiNa^{AT} was stable up to 3 h (FIGS. 6A-6C). Thereafter, fluorescence decreases as RatiNa^{AT} progressively degrades thereby liberating the ATTO dye, which eventually leaches out of the lysosomes. Based on these results, all Na⁺ measurements were conducted within 30 min of RatiNa labeling lysosomes.

RatiNa response was not obscured by autofluorescence in worms or live cells. In the physiologically relevant regime of Na⁺ (5 mM to 145 mM), the performance characteristics of RatiNa in both worms and live cells were similar to those on beads incubated in buffers of known pH and [Na⁺] (FIGS. 2B-2D and 6D-6F). RatiNa response was calibrated by imaging RatiNa-labeled beads in increasing [Na⁺], and RatiNa-labeled worm lysosomes whose lumenal pH and Na⁺ have been clamped using buffers of defined pH and [Na⁺] containing a cocktail of ionophores19 (FIG. 2C). Lysosomal G/R ratios increased linearly with [Na⁺] whether RatiNa was in C. elegans, RAW 264.7 macrophages, or on beads, demonstrating that degradation, if any, is negligible under these conditions (FIG. 5D).

To obtain Na⁺ levels of single lysosomes, heatmaps of Na⁺ were generated from the G/R images of RatiNa-labeled lysosomes in resting cells and compared them to the in vivo or in cellulo calibration profile as relevant (FIGS. 2E and 6G). In both worms and RAW 264.7 macrophages, lumenal Na⁺ varied surprisingly across lysosomes. Na⁺ in single lysosomes ranged between 5 mM to 145 mM, averaging ˜43 mM in C. elegans and ˜48 mM in RAW 264.7 macrophages. This data, which provides single lysosome resolution, reconciles previous population averaged estimates that differed from each other by ˜130 mM7,19.

By mapping Na⁺ as a function of endosomal maturation, RatiNa is shown to capture physiological differences in organellar Na⁺. Because DNA nanodevices are internalized via scavenger receptors into early endosomes that mature to late endosomes and eventually lysosomes34, RatiNa acts as an endocytic tracer, labeling each stage of endosomal maturation as a function of chase time post-injection. These chase times were determined by injecting RatiNa^{AT} into nematodes expressing either the early endosome marker, RAB-5, or the late endosome marker, RAB-729. RatiNa^{AT} localized in early endosomes at 5 min and late endosomes at 17 min post-injection (FIG. 3A) with negligible off-target labeling of other organelles on the endolysosomal pathway (FIGS. 7A-7B). When Nat in early endosomes and late endosomes were measured using RatiNa as described above for lysosomes, lumenal Na⁺ was found to be highest in early endosomes (˜74 mM) and drops to ˜51 mM in late endosomes (FIG. 3B). The high Na⁺ levels in early endosomes might explain their high membrane potential6. Notably, because every other ion mapped so far (i.e., H⁺, Cl⁻ or Ca²⁺) increases progressively along the endolysosomal pathway22,23,29, the data reveals that uniquely, Na⁺ levels decrease as a function of endosomal maturation, implicating Na⁺ efflux mechanisms for every endosomal stage.

RatiNa could also capture physiological differences in Na⁺ arising from the activity of a Na⁺ channel or transporter in a specific organelle. Two-pore channel type 2 (TPC2) is a lysosomal membrane protein that can function as an NAADP activated Ca²⁺ channel or a PI (3,5) P2 activated Na⁺ channel7,35. Depleting PI (3,5) P2 blocks the Na⁺ channel activity of TPC2 and prevents lysosomal Na⁺ export36. RAW 264.7 macrophages treated with apilimod, a specific PIKfyve inhibitor37, depletes PI (3,5) P2 and swells lysosomes38,39. Interestingly, RatiNa revealed that lysosomal Na⁺ increased considerably from ˜48 mM in native lysosomes to ˜70 mM upon PIKfyve inhibition (FIG. 5C). Since Na⁺ levels in late endosomes (˜51 mM) are only marginally higher than those in lysosomes, the data indicates that Nat is elevated in the PIKfyve inhibited lysosomes because Na⁺ release is impacted, likely via TPC2 inhibition, and not due to fusion with late endosomes that contain more Na⁺.

RatiNa was used to map Na⁺ transport in lysosomes of C. elegans under salt stress. Excess salinity stresses many species, including C. elegans, because it interferes with osmoregulation40,41. While salt stress has been studied at cellular resolution in whole animals42, it is not known whether this stress is transferred downstream across intracellular membranes to impact sub-cellular Na⁺ levels. C. elegans adapts to salt stress by increasing glycerol and sorbitol synthesis and regulating its body volume41,42. A well-established assay41 was used to adapt C. elegans to high Na⁺ (FIG. 15). Briefly, worm eggs of the relevant genetic background were grown at either normal salinity (50 mM NaCl, unadapted) or at elevated salinity (200 mM, adapted). Then at the L4 stage larvae were transferred and grown in progressively higher levels of Na⁺ up to a maximum of 400 mM Na⁺. Brood sizes of the adapted worms at each salt concentration were measured and compared to those of unadapted worms. At 400 mM Na⁺, only wild type (N2) worms previously adapted to salt stress produced progeny.

When the assay was repeated on worms lacking various organellar Na⁺ transporters, it was found that impairing Na⁺ mobilization in organelles impacted salt adaptation. Mutant worms lacking various electroneutral Na⁺/H⁺ exchangers (NHX) 43, which transport Na⁺ in exchange for H⁺ across biological membranes, did not produce any progeny at 400 mM Na⁺, indicating a reduced ability to adapt (FIGS. 4A and 8A). Worms lacking NHX-5, the homolog of human NHE9, were the most severely affected, failing to produce progeny even at 300 mM Na⁺. In humans, NHE9 resides in late endosomes and is genetically linked to autism44,45. To map organelle participation in adaptation, RatiNa was used to measure lumenal Na⁺ in early endosomes (EE), late endosomes (LE), and lysosomes (LY) in NHX-5 mutants. While Na⁺ levels in EE and LE of NHX-5 mutants were comparable to those in N2 worms, their lysosomal Na⁺ levels were significantly lower (FIGS. 4B and 8A-8C). Lysosomal Na⁺ levels in NHX-8 and NHX-7 mutants were not significantly altered, demonstrating that NHX-5 specifically facilitates lysosomal Na⁺ import (FIG. 4C). NHX-8 and NHX-7 are worm homologs of mammalian NHE-8 and NHE-2 that reside on the Golgi and plasma membrane, respectively46,47. Importantly, adaptation is compromised in worms lacking either plasma membrane or organellar Na⁺ transporters, revealing that even in metazoans, organelles participate in Na⁺ homeostasis.

When lumenal Na⁺ was mapped along the endolysosomal pathway in adapted N2 worms, it was found that Na⁺ levels in EEs, LEs and LYs were all lowered upon salt adaptation. However, the effect was most pronounced in lysosomes, which showed ˜67% decrease (FIG. 4D). Interestingly, in the few NHX-5 mutants that survive adaptation, Na⁺ levels in EEs and LEs were similar to their unadapted counterparts. Yet, lysosomal Na⁺ in these adapted NHX-5 mutants increased by ˜8-fold, despite no change in the corresponding mRNA levels (FIGS. 4E and 9A). Lysosomal Na⁺ mildly increases even when Δnhx-7, Δnhx-8 or Δncx-2 mutants adapt (FIGS. 9A-9D). Together, the data show that organelles modulate their Na⁺ levels as worms adapt to salinity, with the biggest changes occurring in lysosomes. These changes likely reflect both osmotic stress and Na⁺ stress48, which favour more inert osmolytes like glycerol over Na⁺ in the worm body, and possibly also in the lysosome. Since exchangers like NHX-5 work bi-directionally49, they can either import or export Na⁺ from organelles depending on cellular demand. These data suggest that during salt stress, NHX-5 in N2 worms likely switches from lysosomal Na⁺ import to one of export to support the metabolic preference for glycerol as an osmolyte. Deleting NHX-5 hampers adaptation because this likely impedes cells extruding excessive Na⁺ from their lysosomes, where it finally accumulates to toxic levels from entry through fluid phase endocytosis.

In summary, a novel pH-independent, ratiometric fluorescent probe that reports absolute concentrations of Na⁺ in acidic organelles with single organelle resolution is presented. Using the probe, it was found that unlike any other ion previously mapped on the endolysosomal pathway, Na⁺ levels decrease as endosomes mature. While lysosomal Na⁺ is comparable on average in both C. elegans and mammalian macrophages, those of single lysosomes within a given system varied considerably, ranging from 5 to 145 mM. These findings suggest that the disparity between previous measures may have been due to the upper or lower limits being enriched in the population7,19. RatiNa could also capture physiological changes in lysosomal Na⁺ levels due to the activity of a lysosomal Nat channel such as TPC2.

Given the ability to map organellar Na⁺ fluxes in vivo, it was found that NHX-5, the worm homolog of the human lysosomal Na⁺/H⁺ exchanger NHE9, facilitates lysosomal Na⁺ import. When worms adapt to salt stress, of all the organelles on the endolysosomal pathway, the biggest decrease in lumenal Na⁺ occurs in the lysosome. Worms lacking Na⁺ transporters such as NHX-5 in lysosomes were the weakest at adaptation. The few NHX-5 mutants that survived salt stress showed abnormally high Na⁺ only in their lysosomes. The studies reveal lysosomes as a critical conduit for Na⁺ homeostasis in metazoans. Until now, vacuolar Na⁺/H⁺ exchangers are known to regulate cytosolic pH and Na⁺ levels in yeast15 during salt stress and counter hypotonic stress in cultured mammalian50,51 cells but the direction of Na⁺ flux across the organelle membranes was unknown. These results suggests that under salt stress, NHX-5 switches its directionality to extrude excess Na⁺ accumulating lysosomes via bulk phase endocytosis.

Adaptation to high Na⁺ requires cells to undergo a metabolic shift to produce organic osmolytes such as sorbitol to increase the internal osmotic pressure5253. Cells upregulate autophagy and lysosomal proteolysis to generate and recycle nutrients that are substrates for osmolyte production pathways54,55. The metabolic shift is supported by numerous nutrient transporters that move these substrates across lysosomal and plasma membranes into the cytosol56. Many nutrient transporters move their substrates across membranes by co-transporting Na⁺ and thereby leveraging the transmembrane Na⁺ gradient57. Many of these nutrient transporters and their regulators, such as mTOR, reside on the lysosomal membrane58,59. Thus, lysosomes are a hub that supports this metabolic shift60. This provides a rationale for the large Na⁺ flux across lysosomes during adaptation to salt stress. This newfound ability to map organellar Na⁺ in vivo can illuminate mechanisms of Na⁺ homeostasis at an entirely new level of cellular detail.

Various aspects of the disclosure are further exemplified by the non-limiting embodiments recited in the claims below. In each case, features of multiple claims can be combined in any fashion not inconsistent with the specification and not logically inconsistent.

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

Additional aspects of the present disclosure are provided by the following enumerated embodiments, which may be combined in any number and in any combination that is not logically or technically inconsistent.

Embodiment 1. A method for determining a Na⁺ concentration in a sample comprising:
providing a nucleic acid complex comprising
   a first single-stranded nucleic acid molecule comprising a Na⁺ fluorophore linked thereto, wherein the intensity of the fluorescence of the Na⁺ fluorophore is related to the Na⁺ concentration; and
   a second single-stranded nucleic acid molecule that is partially or fully complementary to the first single-stranded molecule,
measuring an intensity of the Na⁺ fluorophore fluorescence; and
   determining the Na⁺ concentration from the intensity.

Embodiment 2. The method of embodiment 1, wherein the sample is a biological sample selected from a cell, cell extract, cell lysate, tissue, tissue extract, bodily fluid, serum, blood, and blood product.

Embodiment 3. The method of embodiment 1, wherein the sample is a live cell.

Embodiment 4. The method of any of embodiments 1-3, wherein determining is in early endosome, late endosome, plasma membrane, lysosome, autophagolysosome, recycling endosome, cis Golgi network, trans Golgi network, endoplasmic reticulum, peroxisomes, or secretory vesicles.

Embodiment 5. The method of any of embodiments 1-4, wherein the nucleic acid complex further comprises a reference fluorophore linked to the first single-stranded nucleic acid molecule or the second single-stranded nucleic acid molecule.

Embodiment 6. The method of any of embodiments 1-4, wherein the nucleic acid complex further comprises a reference fluorophore linked to the second single-stranded nucleic acid.

Embodiment 7. The method of embodiment 5 or embodiment 6, wherein the reference fluorophore comprises an Atto dye, an Alexa Fluor® dye, a Cy® dye, or a BODIPY dye.

Embodiment 8. The method of claim 7, wherein the reference fluorophore comprises an ATTO647 fluorophore.

Embodiment 9. The method of any of embodiments 5-8, wherein the reference fluorophore is pH insensitive and Na⁺ concentration insensitive, within physiological ranges.

Embodiment 10. The method of any of embodiments 5-9, wherein the method further comprises measuring the intensity of the reference fluorescence of the reference fluorophore, and normalizing the Na⁺ fluorescence to the reference fluorescence.

Embodiment 11. The method of any of embodiments 1-10, wherein the Na⁺ fluorophore is pH insensitive.

Embodiment 12. The method of any of embodiments 1-11, wherein the Na⁺ fluorophore comprises a 1-aza-15-crown-5 ether moiety.

Embodiment 13. The method of any of embodiments 1-12, wherein the Na⁺ fluorophore comprises:

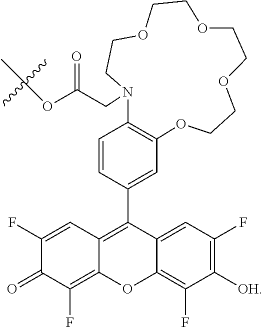 [SMILES: *OC(=O)CN1CCOCCOCCOCCOc2cc(-c3c4cc(F)c(=O)c(F)c-4oc4c(F)c(O)c(F)cc34)ccc21]

Embodiment 14. The method of any of embodiments 1-13, wherein the Na⁺ fluorophore is linked to the first single-stranded nucleic acid through a linker moiety stable under physiological conditions.

Embodiment 15. The method of any of embodiments 1-14, wherein the Na⁺ fluorophore is linked to the first single-stranded nucleic acid molecule through a triazole, thioether, or alkenyl sulfide group.

Embodiment 16. The method any of embodiments 1-15, wherein the Na⁺ fluorophore further comprises a linker moiety configured to form the triazole, thioether, or alkenyl sulfide group through a reaction of an azide, alkyne, or thiol moiety on the Na⁺ fluorophore and an azide, alkyne or alkene moiety on the first single-stranded nucleic acid molecule, as chemically appropriate.

Embodiment 17. The method of any of embodiments 1-16, wherein the Na⁺ fluorophore comprises:

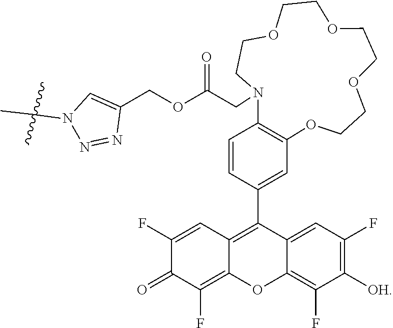 [SMILES: *n1cc(COC(=O)CN2CCOCCOCCOCCOc3cc(-c4c5cc(F)c(=O)c(F)c-5oc5c(F)c(O)c(F)cc45)ccc32)nn1]

Embodiment 18. The method of any of embodiments 1-17, wherein the nucleic acid complex further comprises a third single-stranded nucleic acid molecule that is partially complementary to the second single-stranded molecule.

Embodiment 19. The method of any of embodiments 1-18, wherein the nucleic acid complex further comprises a targeting moiety.

Embodiment 20. The method of embodiment 19, wherein the targeting moiety is a nucleic acid sequence.

Embodiment 21. The method of embodiment 19, wherein the targeting moiety has a cognate artificial protein receptor.

Embodiment 22. The method of any of embodiments 19-21, wherein the targeting moiety is selected from an aptamer, a duplex domain targeted to an artificial protein receptor, a nucleic acid sequence that binds an anionic-ligand binding receptor, and an endocytic ligand.

Embodiment 23. The method of any of embodiments 19-21, wherein the targeting moiety comprises a peptide directly or indirectly conjugated to the nucleic acid molecule.

Embodiment 24. The method of any of embodiments 19-21, wherein the targeting moiety comprises one or more of a fusogenic peptide, a membrane-permeabilizing peptide, a sub-cellular localization sequence, or a cell-receptor ligand.

Embodiment 25. The method of embodiment 24, wherein the targeting moiety comprises a sub-cellular localization sequence, and the sub-cellular localization sequence targets the nucleic acid complex to a region of a cell where spatial localization of a targeted protein is present.

Embodiment 26. The method of embodiment 25, wherein the sub-cellular localization sequence targets the nucleic acid complex to a region of the cell selected from the group consisting of: the cytosol, the endoplasmic reticulum, the mitochondrial matrix, the chloroplast lumen, the medial trans-Golgi cistemae, the lumen of lysosome, the lumen of an endosome, the peroxisome, the nucleus, and a specific spatial location on the plasma membrane.

Embodiment 27. The method of any of embodiments 19-26, wherein the targeting moiety is encoded on the same nucleic acid strand as the first single-stranded nucleic acid molecule, the second single-stranded nucleic acid molecule, the third single-stranded nucleic acid molecule, or any combination thereof.

Embodiment 28. The method of embodiment 27, wherein the targeting moiety is located on the third single-stranded nucleic acid molecule.

Embodiment 29. The method of any of embodiments 1-28, wherein the first and/or second single-stranded nucleic acid molecule is less than 200 nucleotides; or less than 100 nucleotides; or less than 50 nucleotides.

Embodiment 30. The method of any of embodiments 1-29, wherein the first and third single-stranded nucleic molecules together are the same length as the second single-stranded nucleic acid molecule.

Embodiment 31. The method of any of embodiments 1-30, wherein the determined Na⁺ concentration is in a range of 10 μM to 500 mM.

Embodiment 32. The method of any of embodiments 1-31, wherein the determined Na⁺ concentration is in a range of 100 μM to 150 mM.

Embodiment 33. The method of any of embodiments 1-32, wherein the determined Na⁺ concentration is in a range of 1 mM to 150 mM.

Embodiment 34. The method of any of embodiments 1-33, wherein the first single-stranded nucleic acid molecule has the sequence 5′-CG-ATC AAC ACT GCA TAT ATA TAC GAC C-3′ [SEQ ID NO:1] or 5′-ATC AAC ACT GCA TAT ATA TAC GAC C-3′ [SEQ ID NO:2]; and the second single-stranded nucleic acid molecule has the sequence 5′-ATTO647N—C ACT GCA CAC CAG ACA GCA A G GTC GTA TAT ATA TGC AGT GTT GAT-3′ [SEQ ID NO:3].

Embodiment 35. The method of embodiment 34, wherein the nucleic acid complex further comprises a third single-stranded nucleic acid molecule that is partially complementary to the second single-stranded molecule, and wherein the third single-stranded nucleic acid molecule has the sequence 5′-T TGC TGT CTG GTG TGC AGT G-BioTEG-3′ [SEQ ID NO:4] or 5′-T TGC TGT CTG GTG TGC AGT G-3′ [SEQ ID NO:5], wherein bioTEG is a biotin-triethylene glycol moiety.

Embodiment 36. A nucleic acid complex comprising:
a first single-stranded nucleic acid molecule comprising a Na⁺ fluorophore linked thereto, wherein the intensity of the fluorescence of the Na⁺ fluorophore is related to the Na⁺ concentration; and
a second single-stranded nucleic acid molecule that is partially or fully complementary to the first single-stranded molecule.

Embodiment 37. The complex of embodiment 36, wherein the nucleic acid complex further comprises a reference fluorophore linked to the first single-stranded nucleic acid molecule or the second single-stranded nucleic acid molecule.

Embodiment 38. The complex of embodiment 36 or embodiment 37, wherein the nucleic acid complex further comprises a reference fluorophore linked to the second single-stranded nucleic acid.

Embodiment 39. The complex of embodiment 37 or embodiment 38, wherein the reference fluorophore comprises an Atto dye, an Alexa Flour® dye, a Cy® dye, or a BODIPY dye.

Embodiment 40. The complex of embodiment 39, wherein the reference fluorophore comprises an ATTO647 fluorophore.

Embodiment 41. The complex of any of embodiments 37-40, wherein the reference fluorophore is pH insensitive and Na⁺ ion concentration insensitive, within physiological ranges.

Embodiment 42. The complex of any of embodiments 36-41, wherein the Na⁺ fluorophore is pH insensitive.

Embodiment 43. The complex of any of embodiments 36-42, wherein the Na⁺ fluorophore comprises a 1-aza-15-crown-5 ether moiety.

Embodiment 44. The complex of any of embodiments 36-43, wherein the Na⁺ fluorophore comprises:

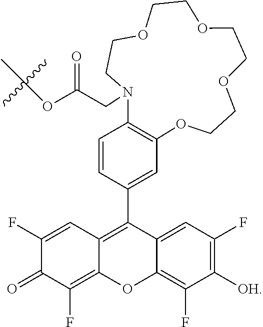 [SMILES: *OC(=O)CN1CCOCCOCCOCCOc2cc(-c3c4cc(F)c(=O)c(F)c-4oc4c(F)c(O)c(F)cc34)ccc21]

Embodiment 45. The complex of any of embodiments 36-44, wherein the Na⁺ fluorophore is linked to the first single-stranded nucleic acid through a linker moiety stable under physiological conditions.

Embodiment 46. The complex of any of embodiments 36-45, wherein the Na⁺ fluorophore is linked to the first single-stranded nucleic acid molecule through a triazole, thioether, or alkenyl sulfide group.

Embodiment 47. The complex any of embodiments 36-46, wherein the Na⁺ fluorophore further comprises a linker moiety configured to form the triazole, thioether, or alkenyl sulfide group through a reaction of an azide, alkyne, or thiol moiety on the Na⁺ fluorophore and an azide, alkyne or alkene moiety on the first single-stranded nucleic acid molecule, as chemically appropriate.

Embodiment 48. The complex of any of embodiments 36-47, wherein the Na⁺ fluorophore comprises:

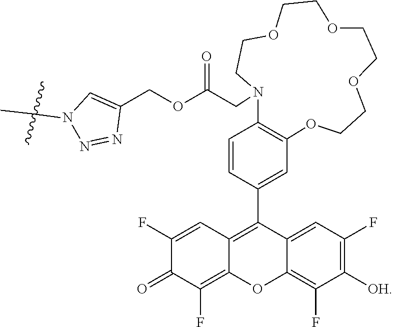 [SMILES: *n1cc(COC(=O)CN2CCOCCOCCOCCOc3cc(-c4c5cc(F)c(=O)c(F)c-5oc5c(F)c(O)c(F)cc45)ccc32)nn1]

Embodiment 49. The complex of any of embodiments 36-48, wherein the nucleic acid complex further comprises a third single-stranded nucleic acid molecule that is partially complementary to the second single-stranded molecule.

Embodiment 50. The complex of any of embodiments 36-49, wherein the nucleic acid complex further comprises a targeting moiety.

Embodiment 51. The complex of embodiment 50, wherein the targeting moiety is a nucleic acid sequence.

Embodiment 52. The complex of embodiment 50, wherein the targeting moiety has a cognate artificial protein receptor.

Embodiment 53. The complex of any of embodiments 50-52, wherein the targeting moiety is encoded on the same nucleic acid strand as the first single-stranded nucleic acid molecule, the second single-stranded nucleic acid molecule, the third single-stranded nucleic acid molecule, or any combination thereof.

Embodiment 54. The complex of any of embodiments 50-52, wherein the targeting moiety is selected from an aptamer, a duplex domain targeted to an artificial protein receptor, a nucleic acid sequence that binds an anionic-ligand binding receptor, and an endocytic ligand.

Embodiment 55. The complex of any of embodiments 50-52, wherein the targeting moiety comprises a peptide directly or indirectly conjugated to the nucleic acid molecule.

Embodiment 56. The complex of any of embodiments 50-52, wherein the targeting moiety comprises one or more of a fusogenic peptide, a membrane-permeabilizing peptide, a sub-cellular localization sequence, or a cell-receptor ligand.

Embodiment 57. The complex of embodiment 56, wherein the targeting moiety comprises a sub-cellular localization sequence, and the sub-cellular localization sequence targets the nucleic acid complex to a region of a cell where spatial localization of a targeted protein is present.

Embodiment 58. The complex of embodiment 57, wherein the sub-cellular localization sequence targets the nucleic acid complex to a region of the cell selected from the group consisting of: the cytosol, the endoplasmic reticulum, the mitochondrial matrix, the chloroplast lumen, the medial trans-Golgi cistemae, the lumen of lysosome, the lumen of an endosome, the peroxisome, the nucleus, and a specific spatial location on the plasma membrane.

Embodiment 59. The complex of any of embodiments 50-58, wherein the targeting moiety is located on the first, second, or third single-stranded nucleic acid molecule.

Embodiment 60. The complex of embodiment 59, wherein the targeting moiety is located on the third single-stranded nucleic acid molecule.

Embodiment 61. The complex of any of embodiments 36-60, wherein the first and/or second single-stranded nucleic acid molecule is less than 200 nucleotides; or less than 100 nucleotides; or less than 50 nucleotides.

Embodiment 62. The complex of any of embodiments 36-61, wherein the first and third single-stranded nucleic molecules together are the same length as the second single-stranded nucleic acid molecule.

Embodiment 63. The complex of any of embodiments 36-62, wherein the determined Na⁺ concentration is in a range of 10 μM to 500 mM.

Embodiment 64. The complex of any of embodiments 36-63, wherein the determined Na⁺ concentration is in a range of 100 μM to 150 mM.

Embodiment 65. The complex of any of embodiments 36-64, wherein the determined Na⁺ concentration is in a range of 1 mM to 150 mM.

Embodiment 66. The complex of any of embodiments 36-65, wherein the first single-stranded nucleic acid molecule has the sequence 5′-CG-ATC AAC ACT GCA TAT ATA TAC GAC C-3′ [SEQ ID NO:1] or 5′-ATC AAC ACT GCA TAT ATA TAC GAC C-3′ [SEQ ID NO:2]; and the second single-stranded nucleic acid molecule has the sequence 5′-ATTO647N—C ACT GCA CAC CAG ACA GCA A G GTC GTA TAT ATA TGC AGT GTT GAT-3′ [SEQ ID NO:3].

Embodiment 67. The complex of embodiment 66, wherein the nucleic acid complex further comprises a third single-stranded nucleic acid molecule that is partially complementary to the second single-stranded molecule, and wherein the third single-stranded nucleic acid molecule has the sequence 5′-T TGC TGT CTG GTG TGC AGT G-BioTEG-3′ [SEQ ID NO:4] or 5′-T TGC TGT CTG GTG TGC AGT G-3′ [SEQ ID NO:5], wherein bioTEG is a biotin-triethylene glycol moiety.

Embodiment 68. A compound having the structure:

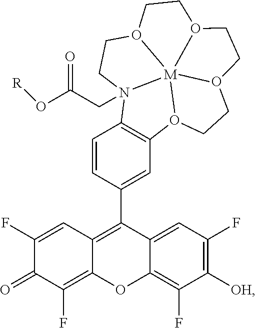

wherein:
M is an alkali metal ion, or is absent; and
R is H, C₁-C₁₂ alkyl, (C₁-C₁₂ alkyl)-SH, (C₁-C₁₂ alkyl)-N₃, C₂-C₁₂ alkenyl group, or C₂-C₁₂ alkynyl.

Embodiment 69. The compound of embodiment 68, wherein M is Na⁺ or absent.

Embodiment 70. The compound of embodiment 68 or embodiment 69, wherein R is C₂-C₆ alkynyl.

Embodiment 71. The compound of any of embodiments 68-70, wherein R is —CH₂—C≡CH.

### REFERENCES

1. Chatton, J.-Y., Magistretti, P. J. & Barros, L. F. Sodium signaling and astrocyte energy metabolism. Glia 64, 1667-1676 (2016).
2. Adrogué, H. J. & Madias, N. E. Sodium and potassium in the pathogenesis of hypertension. N. Engl. J. Med. 356, 1966-1978 (2007).
3. Erecińska, M. & Silver, I. A. Ions and energy in mammalian brain. Prog. Neurobiol. 43, 37-71 (1994).
4. Lamason, R. L. et al. SLC24A5, a putative cation exchanger, affects pigmentation in zebrafish and humans. Science 310, 1782-1786 (2005).
5. Fukada-Tanaka, S., Inagaki, Y., Yamaguchi, T., Saito, N. & Iida, S. Colour-enhancing protein in blue petals. Nature 407, 581 (2000).
6. Saminathan, A. et al. A DNA-based voltmeter for organelles. Na⁺. Nanotechnol. 16, 96-103 (2021).
7. Wang, X. et al. TPC proteins are phosphoinositide-activated sodium-selective ion channels in endosomes and lysosomes. Cell 151, 372-383 (2012).
8. Kondapalli, K. C., Prasad, H. & Rao, R. An inside job: how endosomal Na(+)/H(+) exchangers link to autism and neurological disease. Front. Cell. Neurosci. 8, 172 (2014).
9. Nakamura, N., Tanaka, S., Teko, Y., Mitsui, K. & Kanazawa, H. Four Na+/H+exchanger isoforms are distributed to Golgi and post-Golgi compartments and are involved in organelle pH regulation. J. Biol. Chem. 280, 1561-1572 (2005).
10. Brett, C. L., Wei, Y., Donowitz, M. & Rao, R. Human Na(+)/H(+) exchanger isoform 6 is found in recycling endosomes of cells, not in mitochondria. Am J Physiol, Cell Physiol 282, C1031-41 (2002).
11. Nass, R., Cunningham, K. W. & Rao, R. Intracellular sequestration of sodium by a novel Na+/H+ exchanger in yeast is enhanced by mutations in the plasma membrane H+-ATPase. Insights into mechanisms of sodium tolerance. J. Biol. Chem. 272, 26145-26152 (1997).
12. Cang, C. et al. mTOR regulates lysosomal ATP-sensitive two-pore Na(+) channels to adapt to metabolic state. Cell 152, 778-790 (2013).
13. Palty, R. et al. NCLX is an essential component of mitochondrial Na+/Ca2+ exchange. Proc Natl Acad Sci USA 107, 436-441 (2010).
14. Shapovalov, G. et al. Organelle membrane derived patches: reshaping classical methods for new targets. Sci. Rep. 7, 14082 (2017).
15. Nass, R. & Rao, R. Novel localization of a Na+/H+ exchanger in a late endosomal compartment of yeast. Implications for vacuole biogenesis. J. Biol. Chem. 273, 21054-21060 (1998).
16. Cang, C., Bekele, B. & Ren, D. The voltage-gated sodium channel TPC1 confers endolysosomal excitability. Na⁺. Chem. Biol. 10, 463-469 (2014).
17. Harootunian, A. T., Kao, J. P., Eckert, B. K. & Tsien, R. Y. Fluorescence ratio imaging of cytosolic free Na+ in individual fibroblasts and lymphocytes. J. Biol. Chem. 264, 19458-19467 (1989).
18. Minta, A. & Tsien, R. Y. Fluorescent indicators for cytosolic sodium. J. Biol. Chem. 264, 19449-19457 (1989).
19. Steinberg, B. E. et al. A cation counterflux supports lysosomal acidification. J. Cell Biol. 189, 1171-1186 (2010).
20. Smith, N. K. & Cameron, I. L. Observations on electron probe x-ray microanalysis compared to other methods for measuring intracellular elemental concentration. Scan. Electron Microsc. 395-408 (1981).
21. Modi, S. et al. A DNA nanomachine that maps spatial and temporal pH changes inside living cells. Na⁺. Nanotechnol. 4, 325-330 (2009).
22. Saha, S., Prakash, V., Halder, S., Chakraborty, K. & Krishnan, Y. A pH-independent DNA nanodevice for quantifying chloride transport in organelles of living cells. Na⁺. Nanotechnol. 10, 645-651 (2015).
23. Narayanaswamy, N. et al. A pH-correctable, DNA-based fluorescent reporter for organellar calcium. Na⁺. Methods 16, 95-102 (2019).
24. Martin, V. V., Rothe, A. & Gee, K. R. Fluorescent metal ion indicators based on benzoannelated crown systems: a green fluorescent indicator for intracellular sodium ions. Bioorg. Med. Chem. Lett. 15, 1851-1855 (2005).
25. Holmehave, J., Pedersen, S. K., Jensen, H. & Ogilby, P. R. Aarhus green: a tetrafluoro-substituted derivative of fluorescein. Arkivoc 2015, 52 (2015).
26. Kolb, H. C., Finn, M. G. & Sharpless, K. B. Click Chemistry: Diverse Chemical Function from a Few Good Reactions. Angew. Chem. Int. Ed. 40, 2004-2021 (2001).
27. Rostovtsev, V. V., Green, L. G., Fokin, V. V. & Sharpless, K. B. A stepwise huisgen cycloaddition process: copper (I)-catalyzed regioselective “ligation” of azides and terminal alkynes. Angew. Chem. Int. Ed 41, 2596-2599 (2002).
28. Veetil, A. T. et al. DNA-based fluorescent probes of NOS2 activity in live brains. Proc Natl Acad Sci USA 117, 14694-14702 (2020).
29. Surana, S., Bhat, J. M., Koushika, S. P. & Krishnan, Y. An autonomous DNA nanomachine maps spatiotemporal pH changes in a multicellular living organism. Na⁺. Commun. 2, 340 (2011).
30. Suresh, B. et al. Tubular lysosomes harbor active ion gradients and poise macrophages for phagocytosis. Proc Natl Acad Sci USA 118, (2021).
31. Cui, C. et al. A lysosome-targeted DNA nanodevice selectively targets macrophages to attenuate tumours. Na⁺. Nanotechnol. 16, 1394-1402 (2021).
32. Dan, K., Veetil, A. T., Chakraborty, K. & Krishnan, Y. DNA nanodevices map enzymatic activity in organelles. Na⁺. Nanotechnol. 14, 252-259 (2019).
33. Surana, S., Bhatia, D. & Krishnan, Y. A method to study in vivo stability of DNA nanostructures. Methods 64, 94-100 (2013).
34. Chakraborty, K., Leung, K. & Krishnan, Y. High lumenal chloride in the lysosome is critical for lysosome function. eLife 6, e28862 (2017).
35. Calcraft, P. J. et al. NAADP mobilizes calcium from acidic organelles through two-pore channels. Nature 459, 596-600 (2009).
36. Du, C., Guan, X. & Yan, J. Two-pore channel blockade by phosphoinositide kinase inhibitors YM201636 and PI-103 determined by a histidine residue near pore-entrance. Commun. Biol. 5, 738 (2022).
37. Kang, Y.-L. et al. Inhibition of PIKfyve kinase prevents infection by Zaire ebolavirus and SARS-COV-2. Proc Natl Acad Sci USA 117, 20803-20813 (2020).
38. Choy, C. H. et al. Lysosome enlargement during inhibition of the lipid kinase PIKfyve proceeds through lysosome coalescence. J. Cell Sci. 131, (2018).
39. Leray, X. et al. Tonic inhibition of the chloride/proton antiporter CIC-7 by PI (3,5) P2 is crucial for lysosomal pH maintenance. eLife 11, (2022).
40. Yang, Y. & Guo, Y. Unraveling salt stress signaling in plants. J. Integr. Plant Biol. 60, 796-804 (2018).
41. Lamitina, S. T., Morrison, R., Moeckel, G. W. & Strange, K. Adaptation of the nematode Caenorhabditis elegans to extreme osmotic stress. Am J Physiol, Cell Physiol 286, C785-91 (2004).
42. Urso, S. J. & Lamitina, T. The C. elegans Hypertonic Stress Response: Big Insights from Shrinking Worms. Cell. Physiol. Biochem. 55, 89-105 (2021).
43. Nehrke, K. & Melvin, J. E. The NHX family of Na+-H+ exchangers in Caenorhabditis elegans. J. Biol. Chem. 277, 29036-29044 (2002).
44. Kondapalli, K. C. et al. Functional evaluation of autism-associated mutations in NHE9. Na⁺. Commun. 4, 2510 (2013).
45. Morrow, E. M. et al. Identifying autism loci and genes by tracing recent shared ancestry. Science 321, 218-223 (2008).
46. Collins, J. F. et al. Molecular cloning, sequencing, tissue distribution, and functional expression of a Na+/H+ exchanger (NHE-2). Proc Natl Acad Sci USA 90, 3938-3942 (1993).
47. Oberheide, K., Puchkov, D. & Jentsch, T. J. Loss of the Na+/H+ exchanger NHE8 causes male infertility in mice by disrupting acrosome formation. J. Biol. Chem. 292, 10845-10854 (2017).
48. Assaha, D. V. M., Ueda, A., Saneoka, H., Al-Yahyai, R. & Yaish, M. W. The role of na+ and K+ transporters in salt stress adaptation in glycophytes. Front. Physiol. 8, 509 (2017).
49. Mager, T., Rimon, A., Padan, E. & Fendler, K. Transport mechanism and pH regulation of the Na+/H+ antiporter NhaA from Escherichia coli: an electrophysiological study. J. Biol. Chem. 286, 23570-23581 (2011).
50. Platt, C. D. et al. Leucine-rich repeat containing 8A (LRRC8A)-dependent volume-regulated anion channel activity is dispensable for T-cell development and function. J. Allergy Clin. Immunol. 140, 1651-1659.el (2017).
51. López-Hernández, T., Puchkov, D., Krause, E., Maritzen, T. & Haucke, V. Endocytic regulation of cellular ion homeostasis controls lysosome biogenesis. Na⁺. Cell Biol. 22, 815-827 (2020).
52. Burg, M. B., Ferraris, J. D. & Dmitrieva, N. I. Cellular response to hyperosmotic stresses. Physiol. Rev. 87, 1441-1474 (2007).
53. Jeffery, J. & Jörnvall, H. Enzyme relationships in a sorbitol pathway that bypasses glycolysis and pentose phosphates in glucose metabolism. Proc Natl Acad Sci USA 80, 901-905 (1983).
54. Galluzzi, L., Pietrocola, F., Levine, B. & Kroemer, G. Metabolic control of autophagy. Cell 159, 1263-1276 (2014).
55. Skou, J. C. The Identification of the Sodium-Potassium Pump (Nobel Lecture). Angew. Chem. Int. Ed 37, 2320-2328 (1998).
56. Kandasamy, P., Gyimesi, G., Kanai, Y. & Hediger, M. A. Amino acid transporters revisited: New views in health and disease. Trends Biochem. Sci. 43, 752-789 (2018).
57. Rebsamen, M. et al. SLC38A9 is a component of the lysosomal amino acid sensing machinery that controls mTORC1. Nature 519, 477-481 (2015).
58. Wang, S. et al. Metabolism. Lysosomal amino acid transporter SLC38A9 signals arginine sufficiency to mTORC1. Science 347, 188-194 (2015).
59. Efeyan, A., Comb, W. C. & Sabatini, D. M. Nutrient-sensing mechanisms and pathways. Nature 517, 302-310 (2015).
60. Lamming, D. W. & Bar-Peled, L. Lysosome: The metabolic signaling hub. Traffic 20, 27-38 (2019).
61. Schindelin, J. et al. Fiji: an open-source platform for biological-image analysis. Nat Methods 9, 676-682 (2012).
62. Sorochkina, A. I. et al. N-Terminally Glutamate-Substituted Analogue of Gramicidin A as Protonophore and Selective Mitochondrial Uncoupler. PLOS ONE 7, e41919 (2012).
63. Trivedi, P. C., Bartlett, J. J. & Pulinilkunnil, T. Lysosomal Biology and Function: Modern View of Cellular Debris Bin. Cells 9, 1131 (2020).
64. Ishiguro, H., Steward, M. C., Lindsay, A. R. & Case, R. M. Accumulation of intracellular HCO3- by Na(+)-HCO3-cotransport in interlobular ducts from guinea-pig pancreas. The Journal of Physiology 495, 169-178 (1996).

What is claimed is:

1. A method for determining a Na⁺ concentration in a sample comprising:
providing a nucleic acid complex comprising:
a first single-stranded nucleic acid molecule comprising a Na⁺ fluorophore linked thereto, wherein the intensity of the fluorescence of the Na⁺ fluorophore is related to the Na⁺ concentration; and
a second single-stranded nucleic acid molecule that is partially or fully complementary to the first single-stranded nucleic acid molecule,
measuring an intensity of the Na⁺ fluorophore fluorescence; and
determining the Na⁺ concentration from the intensity.

2. The method of claim 1, wherein the sample is a biological sample selected from a cell, cell extract, cell lysate, tissue, tissue extract, bodily fluid, serum, blood, and blood product.

3. The method of claim 1, wherein the sample is a live cell.

4. The method of claim 1, wherein determining is in early endosome, late endosome, plasma membrane, lysosome, autophagolysosome, recycling endosome, cis Golgi network, trans Golgi network, endoplasmic reticulum, peroxisomes, or secretory vesicles.

5. The method of claim 1, wherein the nucleic acid complex further comprises a reference fluorophore linked to the first single-stranded nucleic acid molecule or the second single-stranded nucleic acid molecule.

6. The method of claim 1, wherein the nucleic acid complex further comprises a reference fluorophore linked to the second single-stranded nucleic acid molecule.

7. The method of claim 5, wherein the reference fluorophore comprises an Atto dye, an Alexa Fluor® dye, a Cy® dye, or a BODIPY dye.

8. The method of claim 7, wherein the reference fluorophore comprises an ATTO647 fluorophore.

9. The method of claim 5, wherein the reference fluorophore is pH insensitive and Na⁺ concentration insensitive, within physiological ranges.

10. The method of claim 5, wherein the method further comprises measuring the intensity of the fluorescence of the reference fluorophore, and normalizing the Na⁺ fluorescence to the reference fluorescence.

11. The method of claim 1, wherein the Na⁺ fluorophore is pH insensitive.

12. The method of claim 1, wherein the Na⁺ fluorophore comprises a 1-aza-15-crown-5 ether moiety.

13. The method of claim 1, wherein the Na⁺ fluorophore comprises:

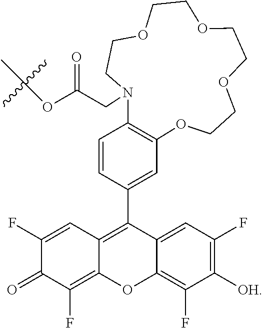 [SMILES: *OC(=O)CN1CCOCCOCCOCCOc2cc(-c3c4cc(F)c(=O)c(F)c-4oc4c(F)c(O)c(F)cc34)ccc21]

14. The method of claim 1, wherein the Na⁺ fluorophore is linked to the first single-stranded nucleic acid molecule through a linker moiety stable under physiological conditions.

15. The method of claim 1, wherein the Na⁺ fluorophore is linked to the first single-stranded nucleic acid molecule through a triazole, thioether, or alkenyl sulfide group.

16. The method claim 1, wherein the Na⁺ fluorophore further comprises a linker moiety configured to form a triazole, thioether, or alkenyl sulfide group through a reaction of an azide, alkyne, or thiol moiety on the Na⁺ fluorophore and an azide, alkyne or alkene moiety on the first single-stranded nucleic acid molecule, as chemically appropriate.

17. The method of claim 1, wherein the Na⁺ fluorophore comprises:

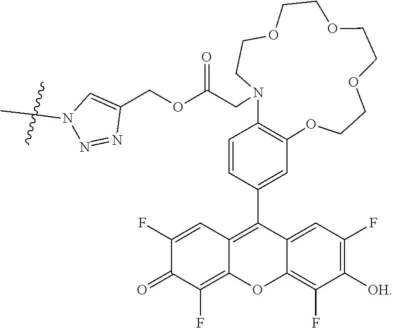 [SMILES: *n1cc(COC(=O)CN2CCOCCOCCOCCOc3cc(-c4c5cc(F)c(=O)c(F)c-5oc5c(F)c(O)c(F)cc45)ccc32)nn1]

18. The method of claim 1, wherein the nucleic acid complex further comprises a third single-stranded nucleic acid molecule that is partially complementary to the second single-stranded nucleic acid molecule.

19. The method of claim 1, wherein the nucleic acid complex further comprises a targeting moiety comprising a nucleic acid sequence or a cognate artificial protein receptor.

20. The method of claim 19, wherein the targeting moiety is selected from an aptamer, a duplex domain targeted to an artificial protein receptor, a nucleic acid sequence that binds an anionic-ligand binding receptor, and an endocytic ligand.

21. The method of claim 19, wherein the targeting moiety comprises a peptide directly or indirectly conjugated to the nucleic acid molecule.

22. The method of claim 19, wherein the targeting moiety comprises one or more of a fusogenic peptide, a membrane-permeabilizing peptide, a sub-cellular localization sequence, or a cell-receptor ligand.

23. The method of claim 22, wherein the targeting moiety comprises a sub-cellular localization sequence, and the sub-cellular localization sequence targets the nucleic acid complex to a region of a cell where spatial localization of a targeted protein is present.

24. The method of claim 23, wherein the sub-cellular localization sequence targets the nucleic acid complex to a region of the cell selected from the group consisting of: the cytosol, the endoplasmic reticulum, the mitochondrial matrix, the chloroplast lumen, the medial trans-Golgi cistemae, the lumen of lysosome, the lumen of an endosome, the peroxisome, the nucleus, and a specific spatial location on the plasma membrane.

25. The method of claim 19, wherein the targeting moiety is encoded on the same nucleic acid strand as the first single-stranded nucleic acid molecule, the second single-stranded nucleic acid molecule, the third single-stranded nucleic acid molecule, or any combination thereof.

26. The method of claim 25, wherein the targeting moiety is located on the third single-stranded nucleic acid molecule.

27. The method of claim 1, wherein the first and/or second single-stranded nucleic acid molecule is less than 200 nucleotides, or less than 100 nucleotides, or less than 50 nucleotides.

28. The method of claim 1, wherein the first and third single-stranded nucleic molecules together are the same length as the second single-stranded nucleic acid molecule.

29. The method of claim 1, wherein the determined Na⁺ concentration is in a range of 10 μM to 500 mM, or in a range of 100 μM to 150 mM, or in a range of 1 mM to 150 mM.

30. The method of claim 1, wherein the first single-stranded nucleic acid molecule has the sequence 5′-CG-ATC AAC ACT GCA TAT ATA TAC GAC C-3′ [SEQ ID NO:1] or 5′-ATC AAC ACT GCA TAT ATA TAC GAC C-3′ [SEQ ID NO:2]; and the second single-stranded nucleic acid molecule has the sequence 5′-ATTO647N—C ACT GCA CAC CAG ACA GCA A G GTC GTA TAT ATA TGC AGT GTT GAT-3′ [SEQ ID NO:3].

31. The method of claim 30, wherein the nucleic acid complex further comprises a third single-stranded nucleic acid molecule that is partially complementary to the second single-stranded nucleic acid molecule, and wherein the third single-stranded nucleic acid molecule has the sequence 5′-T TGC TGT CTG GTG TGC AGT G-BioTEG-3′ [SEQ ID NO:4] or 5′-T TGC TGT CTG GTG TGC AGT G-3′ [SEQ ID NO:5], wherein bioTEG is a biotin-triethylene glycol moiety.

32. A nucleic acid complex comprising:
a first single-stranded nucleic acid molecule comprising a Na⁺ fluorophore linked thereto, wherein the intensity of the fluorescence of the Na⁺ fluorophore is related to the Na⁺ concentration; and
a second single-stranded nucleic acid molecule that is partially or fully complementary to the first single-stranded nucleic acid molecule.


SEQUENCE LISTING (15 sequences)

SEQ ID NO: 1 - DNA, 27 bp, synthetic construct
  source 1..27: mol_type = other DNA
cgatcaacactgcatatatatacgacc

SEQ ID NO: 2 - DNA, 25 bp, synthetic construct
  source 1..25: mol_type = other DNA
atcaacactgcatatatatacgacc

SEQ ID NO: 3 - DNA, 45 bp, synthetic construct
  source 1..45: mol_type = other DNA
  misc_difference 1: note = ATTO647N fluorescent label on 5' end.
cactgcacaccagacagcaaggtcgtatatatatgcagtgttgat

SEQ ID NO: 4 - DNA, 20 bp, synthetic construct
  source 1..20: mol_type = other DNA
  misc_difference 20: note = Biotin triethylene glycol (BioTEG) on the 3' end.
ttgctgtctggtgtgcagtg

SEQ ID NO: 5 - DNA, 20 bp, synthetic construct
  source 1..20: mol_type = other DNA
ttgctgtctggtgtgcagtg

SEQ ID NO: 6 - DNA, 22 bp, synthetic construct
  source 1..22: mol_type = other DNA
cgtcaactgtagcaggttctaa

SEQ ID NO: 7 - DNA, 22 bp, synthetic construct
  source 1..22: mol_type = other DNA
ggaaacgtaggtgaggagtatg

SEQ ID NO: 8 - DNA, 22 bp, synthetic construct
  source 1..22: mol_type = other DNA
ggagctttaccacacgacttat

SEQ ID NO: 9 - DNA, 21 bp, synthetic construct
  source 1..21: mol_type = other DNA
gtgcatgagctgacgaataga

SEQ ID NO: 10 - DNA, 21 bp, synthetic construct
  source 1..21: mol_type = other DNA
ccatcgttcaactcgttacct

SEQ ID NO: 11 - DNA, 21 bp, synthetic construct
  source 1..21: mol_type = other DNA
gagcaatgcactcaacaatcc

SEQ ID NO: 12 - DNA, 24 bp, synthetic construct
  source 1..24: mol_type = other DNA
gattgatcggaggaggagatattg

SEQ ID NO: 13 - DNA, 22 bp, synthetic construct
  source 1..22: mol_type = other DNA
gtagtgagctggatccaagaag

SEQ ID NO: 14 - DNA, 20 bp, synthetic construct
  source 1..20: mol_type = other DNA
cgagcgtggttactctttca

SEQ ID NO: 15 - DNA, 23 bp, synthetic construct
  source 1..23: mol_type = other DNA
cttctgcatacgatcagcaattc